(19) United States
(12) Patent Application Publication
(10) Pub. No.: US 2022/0325218 A1
(43) Pub. Date: Oct. 13, 2022

(54) METHOD OF CONTROLLING GAS FERMENTATION PLATFORM FOR IMPROVED CONVERSION OF CARBON DIOXIDE INTO PRODUCTS

(71) Applicant: LanzaTech, Inc., Skokie, IL (US)

(72) Inventors: Robert John Conrado, Washington, DC (US); Jason Carl Bromley, Chicago, IL (US); Sean Dennis Simpson, Evanston, IL (US); Michael Emerson Martin, Chicago, IL (US)

(21) Appl. No.: 17/658,609

(22) Filed: Apr. 8, 2022

Related U.S. Application Data
(60) Provisional application No. 63/282,546, filed on Nov. 23, 2021.
(60) Provisional application No. 63/173,338, filed on Apr. 9, 2021.
(60) Provisional application No. 63/173,262, filed on Apr. 9, 2021.
(60) Provisional application No. 63/173,247, filed on Apr. 9, 2021.
(60) Provisional application No. 63/173,243, filed on Apr. 9, 2021.

(51) Int. Cl. C12M 1/107; C12P 7/06; C12P 7/16; C12P 7/64; C12P 7/28; C12P 7/08; C12P 7/54; C12N 1/20

(52) U.S. Cl. CPC C12M 21/04; C12P 7/065; C12P 7/16; C12P 7/64; C12P 7/28; C12P 7/08; C12P 7/54; C12N 1/205; C12R 2001/145

(57) ABSTRACT

Methods and systems to control flexible gas fermentation platforms for improved conversion of CO₂ into products is developed and particularly relates to a control process and system to control a ratio of feedstock gases and maximize the concentration of inert components in a bioreactor tail gas stream and or bioreactor headspace. Improved carbon utilization results though providing the most beneficial ratio of substrates to the bioreactor of the fermentation process.

### CROSS REFERENCE TO RELATED APPLICATIONS

This application claims the benefit of U.S. Provisional Patent Application Nos. 63/173,243 filed on Apr. 9, 2021, 63/173,247 filed on Apr. 9, 2021, 63/173,262 filed on Apr. 9, 2021, 63/173,338 filed on Apr. 9, 2021, and 63/282,546 filed on Nov. 23, 2021, the entirety of which is incorporated herein by reference.

### FIELD

This disclosure relates to methods and systems to control flexible fermentation platforms for improved conversion of CO₂ into products. In particular, the disclosure relates to a continuous control process and system to control a ratio of feedstock substrate gasses and maximize the concentration of inert components in an outlet gas stream.

### BACKGROUND

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). The majority of CO₂ comes from the burning fossil fuels to produce energy, although industrial and forestry practices also emit CO₂ into the atmosphere. Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases containing carbon dioxide (CO₂), carbon monoxide (CO), and or hydrogen (H₂), into a variety of fuels and chemicals. Recently, however, gas fermentation has emerged as an alternative platform for the biological fixation of such gases. In particular, C1-fixing microorganisms have been demonstrated to convert gases containing CO₂, CO, and or H₂ such as industrial waste gas or syngas or mixtures thereof into products such as ethanol and 2,3-butanediol. Efficient production of such products may be limited, for example, by slow microbial growth, limited gas uptake, sensitivity to toxins, or diversion of carbon substrates into undesired by-products.

The C1-carbon source may be a waste gas obtained as a by-product of an industrial process or from another source, such as combustion engine exhaust fumes, CO₂ by-product gases from industrial processes (cement production) ammonia production, by-product gas from syngas clean-up, ethylene production, ethylene oxide production, methanol synthesis) off-gas from fermentation processes (such as the conversion of sugar into ethanol), biogas, landfill gas, direct air capture, mined CO₂ (fossil CO₂), or from electrolysis. The C1-carbon source may be syngas generated by pyrolysis, torrefaction, or gasification. In other words, waste material may be recycled by pyrolysis, reforming, torrefaction, or gasification to generate syngas which is used as the substrate and or C1-carbon source.

In certain embodiments, the industrial process is selected from ferrous metal products manufacturing, such as a steel mill manufacturing, non-ferrous products manufacturing, petroleum refining, electric power production, carbon black production, paper and pulp manufacturing, ammonia production, methanol production, coke manufacturing, or any combination thereof. In these embodiments, the substrate and or C1-carbon source may be captured from the industrial process before it is emitted into the atmosphere, using any known method.

The C1-carbon source may be syngas, such as syngas obtained by gasification of coal, gasification of refinery residues, gasification of biomass, gasification of lignocellulosic material, black liquor gasification, gasification of municipal solid waste, gasification of industrial solid waste, gasification of sewerage, gasification of sludge from wastewater treatment, reforming of natural gas, reforming of biogas, reforming of landfill gas, or any combination thereof.

Examples of municipal solid waste include tires, plastics, and fibers in shoes, apparel, textiles. The municipal solid waste may be sorted or unsorted. Examples of biomass may include lignocellulosic material and may also include microbial biomass. Lignocellulosic material may include agriculture waste and forest waste.

The industrial gases or syngas may require treatment or decomposition to be suitable for use in gas fermentation systems. It has been shown that high CO₂ content in the industrial gases or syngas adversely impacts ethanol selectivity benefit of the fermentation and results in higher production of undesired co-products such as acetate, and 2,3-butanediol.

Accordingly, there remains a need for a control process and system for a flexible fermentation platform that can continuously control a ratio of substrate gasses provided to a bioreactor of a fermentation platform to maximize the concentration of inert component in a gas outlet stream of the bioreactor. It is particularly advantageous in situations where an initial starting material is CO₂. Moreover, in some embodiments a need exists to convert some CO₂ present in the syngas or industrial gas to CO prior to introduction into the bioreactor because improved CO content and improved H₂:CO ratio, has shown to improve microorganism growth and stability.

### SUMMARY

The disclosure involves a method for continuously controlling a ratio of input gases provided to a bioreactor of a continuous gas fermentation process comprising: a) providing a gas fermentation process comprising: a first gaseous stream comprising H₂ from a H₂ source; a second gaseous stream comprising CO₂ from an industrial or syngas process; a CO₂ to CO conversion zone in fluid communication with the second gaseous stream and optionally the first gaseous stream, and having a CO enriched effluent comprising CO and CO₂; at least one bioreactor having at least one C-1 fixing bacterium for gas fermentation in a nutrient solution, the bioreactor having an product stream comprising at least one product, an outlet gas stream comprising H₂, CO₂, and inert components, a headspace comprising H₂, CO₂, and inert components, or both, the bioreactor in fluid communication with the CO enriched effluent, optionally the first gaseous stream, optionally the second gaseous stream, or any combination thereof; b) measuring a H₂:CO:CO₂ molar ratio of the bioreactor outlet gas stream or the bioreactor headspace, to provide a measured H₂:CO:CO₂ molar ratio; c) inputting the measured H₂:CO:CO₂ molar ratio to a controller and comparing the measured H₂:CO:CO₂ molar ratio to a predetermined H₂:CO:CO₂ molar ratio; and d) adjusting the flowrate of the first gaseous stream, the flowrate of the second gaseous stream, or both, in response to the difference between the measured H₂:CO:CO₂ molar ratio and the predetermined H₂:CO:CO₂ molar ratio to maximize the concentration of inert components in the bioreactor outlet gas stream. The method of may further comprise: compressing, in a first compressor, at least a portion of the first gaseous stream, at least a portion of the second gaseous stream, or any combination thereof to generate a compressed first gaseous stream, a compressed second gaseous stream, and/or a compressed combination first gaseous stream and second gaseous stream; treating: at least a portion of the first gaseous stream or the compressed first gaseous stream, or both; and at least a portion of the second gaseous stream or the compressed second gaseous stream, or both; or the compressed combination first gaseous stream and second gaseous stream; in a gas treatment zone comprising a gas component removal unit, a gas desulfurization/acid gas removal unit, or both before passing the second gaseous stream and optionally the first gaseous stream to the CO₂ to CO conversion zone; and recycling the outlet gas stream to the first compressor, the gas treatment zone, the CO₂ to CO conversion system, the first gaseous stream, the second gaseous stream, or the combination of the first gaseous stream and the second gaseous stream. The method may further comprise combining with the CO enriched effluent stream, at least a portion of: the treated stream; or the first gaseous stream; or the second gaseous stream; or the combination of the first gaseous stream and the second gaseous stream; or the compressed first gaseous stream; or the compressed second gaseous stream; or the compressed combination first gaseous stream and second gaseous stream; or any combination thereof. The first gaseous stream comprising H₂ may be passed to the bioreactor without passing through the CO₂ to CO conversion zone, the method further comprising: compressing the bioreactor outlet gas stream to generate a compressed bioreactor outlet gas stream; passing at least a first portion of the compressed bioreactor outlet gas stream, in any order, to: a gas desulfurization and/or acid gas removal unit; or a gas component removal unit; or both the gas desulfurization and/or acid gas removal unit and the gas component removal unit; to generate a compressed treated bioreactor outlet gas stream; recycling the compressed treated bioreactor outlet gas stream: to combine with the first gaseous stream, the second gaseous stream, or a combination thereof; or to the CO₂ to CO conversion system; or to combine with the CO enriched effluent stream; or any combination thereof; and optionally recycling a second portion of the compressed bioreactor outlet gas stream to combine with the CO enriched effluent stream or to the bioreactor. The method may further comprise combining at least a second portion of the first gaseous stream, at least a second portion of the second gaseous stream, or a combination thereof, with the CO enriched effluent stream. The method may further comprise passing at least at least a second portion of the first gaseous stream, at least a second portion of the second gaseous stream, or a combination thereof, to the bioreactor. The method may further comprise compressing any portions of the first gaseous stream, the second gaseous stream, or combinations thereof. The method may further comprise controlling the relative amounts of the first portion of the compressed outlet gas stream and the second portion of the compressed outlet gas stream using a control valve. The method may further comprise passing at least a portion of the outlet gas stream to an outlet gas CO₂ to CO conversion system selected from a reverse water gas shift unit, a thermo-catalytic conversion unit, a partial combustion unit, a plasma conversion unit, a gasification unit, or a reforming unit, to generate a CO enriched effluent stream and recycling the second CO enriched effluent stream to the bioreactor. The CO enriched effluent stream may comprise a H₂:CO:CO₂ molar ratio of about 5:1:1, about 4.5:1:1, about 4.33:1:1, about 3:1:1, about 2:1:1, about 1:1:1; or about 1:3:1. The CO₂ to CO conversion system may comprise at least one of a reverse water gas shift unit, a thermo-catalytic conversion unit, a partial combustion unit, a reforming unit, or a plasma conversion unit. The product stream may comprise at least one fermentation product selected from ethanol, acetate, butanol, butyrate, 2,3-butanediol, lactate, butene, butadiene, methyl ethyl ketone, ethylene, acetone, isopropanol, lipids, 3-hydroxypropionate, isoprene, fatty acids, 2-butanol, 1,2-propanediol, hexanol, octanol, or 1-propanol. The hydrogen source may comprise at least one of a water electrolyser, a hydrocarbon reforming source, a hydrogen purification source, a solid biomass gasification source, a solid waste gasification source, a coal gasification source, a hydrocarbon gasification source, a methane pyrolysis source, a refinery tail gas production source, a plasma reforming reactor, partial oxidation reactor, or any combination thereof. The industrial or syngas process may be selected from at least one of a sugar-based ethanol production source, a first generation corn-ethanol production source, a second generation corn-ethanol production source, a sugarcane ethanol production source, a cane sugar ethanol production source, a sugar beet ethanol production source, a molasses ethanol production source, a wheat ethanol production source, a grain based ethanol production source, a starch based ethanol production source, a cellulosic based ethanol production source, a cement production source, a methanol synthesis source, an olefin production source, a steel production source, a ferroalloy production source, a refinery tail gas production source, a post combustion gas production source, a biogas production source, a landfill production source, an ethylene oxide production source, a methanol production source, an ammonia production source, mined CO₂ production source, natural gas processing production source, a gasification source, an organic waste gasification source, direct air capture, or any combination thereof. The at least one Cl fixing bacterium may be selected from Clostridium autoethanogenum, Clostridium ljungdahlii, or Clostridium ragsdalei.

The disclosure involves a system for controlling a ratio of substrate gases provided to a bioreactor of a continuous gas fermentation process comprising: a) a first gaseous stream comprising substrate H₂ from a H₂ source; b) a second gaseous stream comprising substrate CO₂ from an industrial or syngas process; c) a CO₂ to CO conversion zone in fluid communication with the second gaseous stream and optionally the first gaseous stream, and having an effluent comprising CO and CO₂; d) at least one bioreactor having at least one C-1 fixing bacterium for gas fermentation in a nutrient solution, the bioreactor having an tail gas stream comprising H₂, CO₂, and inert components, a headspace comprising H₂, CO₂, and inert components, or both, the bioreactor in fluid communication with the effluent comprising CO and CO₂, optionally the first gaseous stream, optionally the second gaseous stream, or any combination thereof; e) sensors in the bioreactor tails gas stream or in the bioreactor headspace or both, capable of measuring the H₂:CO₂ molar ratio or the H₂:CO:CO₂ molar ratio of the bioreactor tail gas stream, or the bioreactor headspace, and providing a measured H₂:CO₂ molar ratio or a measured H₂:CO:CO₂ molar ratio; f) a controller configured to accept inputs of the measured H₂:CO₂ molar ratio or the measured H₂:CO:CO₂ molar ratio and compare the measured H₂:CO₂ molar ratio to a predetermined H₂:CO₂ molar ratio or compare the measured H₂:CO:CO₂ molar ratio to a predetermined H₂:CO:CO₂ molar ratio; and provide outputs to adjust the flowrate of the first gaseous stream, the flowrate of the second gaseous stream, or both, in response to the difference between the measured H₂:CO₂ molar ratio and the predetermined H₂:CO₂ molar ratio or in response to the difference between the in response to the difference between the measured H₂:CO:CO₂ molar ratio and the predetermined H₂:CO:CO₂ molar ratio to maximize the concentration of inert components in the tail gas stream. The system may further comprise outputs to an operating parameter of the CO₂ to CO conversion zone to increase or decrease the relative amount of CO in the effluent comprising CO and CO₂. The CO₂ to CO conversion system may comprise at least one of a reverse water gas shift process, a CO₂ electrolyzer, a thermo-catalytic conversion process, a partial combustion process, or a plasma conversion process. The gas fermentation process may further comprise a gas treatment zone in fluid communication with the first gaseous stream, the second gaseous stream, the effluent, or any combination thereof. The gas fermentation process may further comprise at least one compressor in fluid communication with the first gaseous stream, the second gaseous stream, the effluent, or any combination thereof. The gas fermentation process may further comprise a methane conversion zone in fluid communication with the bioreactor tail gas stream, the methane reforming zone comprising an effluent conduit in fluid communication with the CO₂ to CO conversion zone.

The disclosure involves a control process for an integrated process for the production of at least one fermentation product from a gaseous stream, the control process first comprises providing a gas fermentation process comprising: obtaining a first gaseous stream comprising hydrogen and a second gaseous stream comprising CO₂; passing at least a portion of the first gaseous stream and at least a portion of the second gaseous stream to a CO₂ to CO conversion system operated under conditions to produce a CO enriched exit stream; fermenting the CO enriched exit stream in a bioreactor having a culture of one or more Cl fixing bacterium to produce at least one fermentation product stream and a bioreactor tail gas stream; compressing the bioreactor tail gas stream to generate a compressed bioreactor tail gas stream; passing at least a first portion of the compressed bioreactor tail gas stream, in any order, to: i) a gas desulfurization and or acid gas removal unit; or ii) a gas component removal unit; or iii) both the gas desulfurization and or acid gas removal unit and the gas component removal unit; to generate a compressed treated bioreactor tail gas stream; recycling the compressed treated bioreactor tail gas stream: a) to combine with the first gaseous stream, the second gaseous stream, or a combination thereof; or b) to the CO₂ to CO conversion system; or c) to combine with the CO enriched exit stream; or d) any combination thereof; and optionally recycling a second portion of the compressed bioreactor tail gas stream to combine with the CO enriched exit stream or to the bioreactor. The control process further comprises measuring data to provide the H₂:CO₂ or the H₂:CO:CO₂ molar ratio of the bioreactor tail gas stream, the bioreactor headspace, or both, to provide at least one measured H₂:CO₂ or H₂:CO:CO₂ molar ratio; inputting the measured H₂:CO₂ or H₂:CO:CO₂ molar ratio to a controller and comparing the measured H₂:CO₂ or H₂:CO:CO₂ molar ratio to a predetermined H₂:CO₂ or H₂:CO:CO₂ molar ratio; and adjusting the flowrate of the first gaseous stream, the flowrate of the second gaseous stream, or both, in response to the difference between the measured H₂:CO₂ or H₂:CO:CO₂ molar ratio and the predetermined H₂:CO₂ or H₂:CO:CO₂ molar ratio to maximize the concentration of inert components in the bioreactor tail gas stream, the bioreactor headspace, or both. The inert components may comprise nitrogen or methane or both. A target maximum of the concentration of inert components in the bioreactor tail gas stream or in the bioreactor headspace is from about 70 vol-% to about 80 vol-%.

In the gas fermentation process, at least a second portion of the first gaseous stream, at least a second portion of the second gaseous stream, or a combination thereof, may be combined with the CO enriched exit stream. Any portions of the first gaseous stream, the second gaseous stream, or combinations thereof may be compressed. The relative amounts of the first portion of the compressed tail gas stream and the second portion of the compressed tail gas stream may be controlled using a control valve. At least a portion of the tail gas stream may be passed to a tail gas CO₂ to CO conversion system selected from a reverse water gas shift unit, a thermo-catalytic conversion unit, a partial combustion unit, a plasma conversion unit, a gasification unit, or a reforming unit, to generate a CO enriched effluent stream and the second CO enriched effluent stream may be recycled to the bioreactor. The CO enriched exit stream may comprise a H₂:CO:CO₂ molar ratio of about 5:1:1, about 4.5:1:1, about 4.33:1:1, about 3:1:1, about 2:1:1, about 1:1:1; or about 1:3:1. The CO₂ to CO conversion system may comprises at least one of a reverse water gas shift unit, a thermo-catalytic conversion unit, a partial combustion unit, a reforming unit, or a plasma conversion unit. The at least one fermentation product may be selected from ethanol, acetate, butanol, butyrate, 2,3-butanediol, lactate, butene, butadiene, methyl ethyl ketone, ethylene, acetone, isopropanol, lipids, 3-hydroxypropionate, isoprene, fatty acids, 2-butanol, 1,2-propanediol, hexanol, octanol, or 1-propanol. The first gaseous stream comprising hydrogen may be produced by a hydrogen production source comprising at least one of a water electrolyser, a hydrocarbon reforming source, a hydrogen purification source, a solid biomass gasification source, a solid waste gasification source, a coal gasification source, a hydrocarbon gasification source, a methane pyrolysis source, a refinery tail gas production source, a plasma reforming reactor, partial oxidation reactor, or any combination thereof. The second gaseous stream comprising CO₂ may be produced by a gas production source comprising at least one of a sugar-based ethanol production source, a first generation corn-ethanol production source, a second generation corn-ethanol production source, a sugarcane ethanol production source, a cane sugar ethanol production source, a sugar beet ethanol production source, a molasses ethanol production source, a wheat ethanol production source, a grain based ethanol production source, a starch based ethanol production source, a cellulosic based ethanol production source, a cement production source, a methanol synthesis source, an olefin production source, a steel production source, a ferroalloy production source, a refinery tail gas production source, a post combustion gas production source, a biogas production source, a landfill production source, an ethylene oxide production source, a methanol production source, an ammonia production source, mined CO₂ production source, natural gas processing production source, a gasification source, an organic waste gasification source, direct air capture, or any combination thereof. At least one of the Cl fixing bacterium may be selected from Clostridium autoethanogenum, Clostridium ljungdahlii, or Clostridium ragsdalei.

The disclosure also involves a control process for an integrated process for the production of at least one fermentation product from a gaseous stream, the control process first comprises providing a gas fermentation process comprising: obtaining a first gaseous stream comprising hydrogen and a second gaseous stream comprising CO₂; optionally compressing, in a first compressor, at least a portion of the first gaseous stream, at least a portion of the second gaseous stream, or any combination thereof to generate a compressed first gaseous stream, a compressed second gaseous stream, and or a compressed combination first gaseous stream and second gaseous stream; treating: i) at least a portion of the first gaseous stream or the compressed first gaseous stream, or both; and at least a portion of the second gaseous stream or the compressed second gaseous stream, or both; or ii) the compressed combination first gaseous stream and second gaseous stream; in a gas treatment zone comprising a gas component removal unit, a gas desulfurization/acid gas removal unit, or both to generate a treated stream; converting CO₂ in at least a first portion of the treated stream to form CO in a CO₂ to CO conversion system operated under conditions to produce a CO enriched exit stream; fermenting the CO enriched exit stream in a bioreactor having a culture of one or more Cl fixing bacterium to produce at least one fermentation product stream and a bioreactor tail gas stream; and recycling the tail gas stream to the first compressor, the first gaseous stream, the second gaseous stream, or the combination of the first gaseous stream and the second gaseous stream. The control process further comprises measuring data to provide the H₂:CO₂ or the H₂:CO:CO₂ molar ratio of the bioreactor tail gas stream, the bioreactor headspace, or both, to provide at least one measured H₂:CO₂ or H₂:CO:CO₂ molar ratio; inputting the measured H₂:CO₂ or H₂:CO:CO₂ molar ratio to a controller and comparing the measured H₂:CO₂ or H₂:CO:CO₂ molar ratio to a predetermined H₂:CO₂ or H₂:CO:CO₂ molar ratio; and adjusting the flowrate of the first gaseous stream, the flowrate of the second gaseous stream, or both, in response to the difference between the measured H₂:CO₂ or H₂:CO:CO₂ molar ratio and the predetermined H₂:CO₂ or H₂:CO:CO₂ molar ratio to maximize the concentration of inert components in the bioreactor tail gas stream, the bioreactor headspace, or both. The inert components may comprise nitrogen or methane or both. A target maximum of the concentration of inert components in the bioreactor tail gas stream or in the bioreactor headspace is from about 70 vol-% to about 80 vol-%.

In the gas fermentation process the CO enriched exit stream may be combined with at least a portion of: the treated stream; or the first gaseous stream; or the second gaseous stream; or the combination of the first gaseous stream and the second gaseous stream; or the compressed first gaseous stream; or the compressed second gaseous stream; or the compressed combination first gaseous stream and second gaseous stream; or any combination thereof. At least a portion of the tail gas stream may be passed to a tail gas CO₂ to CO conversion system selected from a reverse water gas shift unit, a thermo-catalytic conversion unit, a partial combustion unit, a plasma conversion unit, a gasification unit, or a reforming unit to generate a CO enriched effluent stream and recycling the second CO enriched effluent stream to the bioreactor. The CO enriched exit stream may further comprise hydrogen and CO₂ and may comprise a H₂:CO:CO₂ molar ratio of about 5:1:1, about 4.5:1:1, about 4.33:1:1, about 3:1:1, about 2:1:1, about 1:1:1; or about 1:3:1. The CO₂ to CO conversion system may comprise at least one of a reverse water gas shift unit, a thermo-catalytic conversion unit, a partial combustion unit, a reforming unit, or a plasma conversion unit. The gas treatment zone may further comprise a deoxygenation unit, a catalytic hydrogenation unit, an adsorption unit, a thermal oxidizer, or any combination thereof. The at least one fermentation product may be selected from ethanol, acetate, butanol, butyrate, 2,3-butanediol, lactate, butene, butadiene, methyl ethyl ketone, ethylene, acetone, isopropanol, lipids, 3-hydroxypropionate, isoprene, fatty acids, 2-butanol, 1,2-propanediol, hexanol, octanol, or 1-propanol. The first gaseous stream comprising hydrogen may be produced by a hydrogen production source discussed above and the second gaseous stream comprising CO₂ may be produced by a gas production source described above. At least one of the Cl fixing bacterium may be selected from Clostridium autoethanogenum, Clostridium ljungdahlii, or Clostridium ragsdalei. The CO enriched exit stream may comprise hydrogen and the process may further comprise separating hydrogen from the CO enriched exit stream and recycling the separated hydrogen to combine with the tail gas stream or to the compressor. The remainder of the CO enriched exit stream may be compressed after the separation of hydrogen. The tail gas stream may comprise methane, the process further comprising passing a portion of the tail gas stream to a methane conversion unit to generate a methane conversion unit effluent and combining the methane conversation unit effluent with the tail gas stream. A stream comprising oxygen may be generated from an oxygen source and passed to the methane conversion unit. A second gaseous stream comprising hydrogen may be passed from the hydrogen source to the bioreactor, a second gaseous stream comprising CO₂ from the CO₂ source may be passed to the bioreactor, or both. A second gaseous stream comprising hydrogen from the hydrogen source may be passed to the bioreactor or combined with the CO enriched exit stream, a second gaseous stream comprising CO₂ from the CO₂ source may be passed to the bioreactor or combined with the CO enriched exit stream, or any combination thereof may be performed. The combining of the second gaseous stream comprising hydrogen from the hydrogen source with the CO enriched exit stream, or the combining of the second gaseous stream comprising CO₂ from the CO₂ source with the CO enriched exit stream, or both may be accomplished by mixing in a mixer. The ratio of the second gaseous stream comprising hydrogen from the hydrogen source to the CO enriched exit stream entering the bioreactor may be from about greater than 0:1 to about 4:1. The CO₂ to CO conversion system may comprise a fired heater having burners and at least a portion of the tail gas stream may be recycled at least to the burners of the fired heater. The CO₂ to CO conversion system may comprises a steam generator producing steam, or a water knockout unit generating a water stream, or both. A portion of CO enriched exit stream may be passed to an inoculator reactor, a buffer tank, or both, and the passing may be directly to the inoculator reactor, buffer tank, or both without intervening units.

The disclosure also involves a method of controlling an integrated process for the production of at least one fermentation product from a gaseous stream, the control process first comprises providing a gas fermentation process comprising: providing a gas fermentation process comprising: obtaining a first gaseous stream comprising hydrogen and a second gaseous stream comprising CO₂; passing at least a portion of the second gaseous stream and optionally a portion of the first gaseous stream to a CO₂ to CO conversion system operated under conditions to produce a CO enriched exit stream; passing at least a portion of the first gaseous stream comprising hydrogen and the CO enriched exit stream to a bioreactor having a culture of one or more Cl fixing bacterium and fermenting to produce at least one fermentation product stream and a bioreactor tail gas stream, the bioreactor optionally having a headspace; compressing the bioreactor tail gas stream to generate a compressed bioreactor tail gas stream; passing at least a first portion of the compressed bioreactor tail gas stream, in any order, to: a gas desulfurization and or acid gas removal unit; or a gas component removal unit; or both the gas desulfurization and or acid gas removal unit and the gas component removal unit; to generate a compressed treated bioreactor tail gas stream; recycling the compressed treated bioreactor tail gas stream: to combine with the first gaseous stream, the second gaseous stream, or a combination thereof; or to the CO₂ to CO conversion system; or to combine with the CO enriched exit stream; or any combination thereof; and optionally recycling a second portion of the compressed bioreactor tail gas stream to combine with the CO enriched exit stream or to the bioreactor. The control process further comprises measuring data to provide the H₂:CO₂ or the H₂:CO:CO₂ molar ratio of the bioreactor tail gas stream, the bioreactor headspace, or both, to provide at least one measured H₂:CO₂ or H₂:CO:CO₂ molar ratio; inputting the measured H₂:CO₂ or H₂:CO:CO₂ molar ratio to a controller and comparing the measured H₂:CO₂ or H₂:CO:CO₂ molar ratio to a predetermined H₂:CO₂ or H₂:CO:CO₂ molar ratio; and adjusting the flowrate of the first gaseous stream, the flowrate of the second gaseous stream, or both, in response to the difference between the measured H₂:CO₂ or H₂:CO:CO₂ molar ratio and the predetermined H₂:CO₂ or H₂:CO:CO₂ molar ratio to maximize the concentration of inert components in the bioreactor tail gas stream, the bioreactor headspace, or both. The inert components may comprise nitrogen or methane or both. A target maximum of the concentration of inert components in the bioreactor tail gas stream or in the bioreactor headspace is from about 70 vol-% to about 80 vol-%.

The disclosure also involves a system for controlling a ratio of substrate gases provided to a bioreactor of a continuous gas fermentation process comprising: a first gaseous stream comprising substrate H₂ from a H₂ source; a second gaseous stream comprising substrate CO₂ from an industrial or syngas process; a CO₂ to CO conversion zone in fluid communication with the second gaseous stream and optionally the first gaseous stream, and having an effluent comprising CO and CO₂; at least one bioreactor having at least one C-1 fixing bacterium for gas fermentation in a nutrient solution, the bioreactor having an tail gas stream comprising H₂, CO₂, and inert components, a headspace comprising H₂, CO₂, and inert components, or both, the bioreactor in fluid communication with the effluent comprising CO and CO₂, optionally the first gaseous stream, optionally the second gaseous stream, or any combination thereof; sensors in the bioreactor tails gas stream or in the bioreactor headspace or both, capable of measuring the H₂:CO₂ molar ratio or the H₂:CO:CO₂ molar ratio of the bioreactor tail gas stream, or the bioreactor headspace, and providing a measured H₂:CO₂ molar ratio or a measured H₂:CO:CO₂ molar ratio; a controller configured to accept inputs of the measured H₂:CO₂ molar ratio or the measured H₂:CO:CO₂ molar ratio and compare the measured H₂:CO₂ molar ratio to a predetermined H₂:CO₂ molar ratio or compare the measured H₂:CO:CO₂ molar ratio to a predetermined H₂:CO:CO₂ molar ratio; and provide outputs to adjust the flowrate of the first gaseous stream, the flowrate of the second gaseous stream, or both, in response to the difference between the measured H₂:CO₂ molar ratio and the predetermined H₂:CO₂ molar ratio or in response to the difference between the in response to the difference between the measured H₂:CO:CO₂ molar ratio and the predetermined H₂:CO:CO₂ molar ratio to maximize the concentration of inert components in the tail gas stream. The system may further comprise outputs to an operating parameter of the CO₂ to CO conversion zone to increase or decrease the relative amount of CO in the effluent comprising CO and CO₂. The CO₂ to CO conversion system of the overall system may comprises at least one of a reverse water gas shift process, a CO₂ electrolyzer, a thermo-catalytic conversion process, a partial combustion process, or a plasma conversion process. The gas fermentation process of the system may further comprise a gas treatment zone in fluid communication with the first gaseous stream, the second gaseous stream, the effluent, or any combination thereof.

### BRIEF DESCRIPTION OF THE DRAWINGS

FIG. 1 shows a flow scheme having a bioreactor, a compressor, a gas treatment zone, a CO₂ to CO conversion system, wherein at least a portion of a bioreactor tail gas from the bioreactor passes through a gas component removal unit, is compressed, and then recycled to the bioreactor, a CO₂ to CO conversion system or both, wherein the flow scheme is controlled by one embodiment of the disclosure.

FIG. 2 shows a flow scheme wherein at least portion of the tail gas from a bioreactor is recycled to the bioreactor, wherein the flow scheme is controlled by one embodiment of the disclosure.

FIG. 3 shows a flow scheme of an embodiment wherein at least portion of the tail gas from a bioreactor is compressed and passed through a gas desulfurization/acid gas removal unit, and then recycled to a CO₂ to CO conversion system, wherein the flow scheme is controlled by one embodiment of the disclosure.

FIG. 4 shows a flow scheme of an embodiment wherein at least a portion of the tail gas from a bioreactor is compressed and passed to an optional controller to split the tail gas stream and optionally recycle a portion to the bioreactor and while passing the remainder of the tail gas to a gas treatment zone. The effluent of the gas treatment zone is recycled to the CO₂ to CO conversion system or upstream of the CO₂ to CO conversion system. The flow scheme is controlled by one embodiment of the disclosure.

FIG. 5 shows a flow scheme of an embodiment similar to that of FIG. 4 with an additional compressor upstream of a CO₂ to CO conversion system. The flow scheme is controlled by one embodiment of the disclosure.

FIG. 6 shows a flow scheme of an embodiment wherein at least a portion of the tail gas stream is recycled to a compressor upstream of a gas treatment zone and CO₂ to CO conversion system. The compressor operates on the combination of a first gaseous stream comprising hydrogen and a second gaseous stream comprising CO₂. The flow scheme is controlled by one embodiment of the disclosure.

FIG. 7 shows a flow scheme of an embodiment similar to that of FIG. 6, except that compressor operates on only a second gaseous stream comprising CO₂ and not on a first gaseous stream comprising hydrogen. The first gaseous stream comprising hydrogen is added to an input stream, the effluent, or both of a gas treatment zone. The flow scheme is controlled by one embodiment of the disclosure.

FIG. 8 shows a flow scheme wherein the compressor operates on only a portion of a second gaseous stream comprising CO₂ and not on a first gaseous stream comprising hydrogen. The remainder of the second gaseous stream comprising CO₂ is not compressed and may be combined with the first gaseous stream comprising hydrogen. The flow scheme is controlled by one embodiment of the disclosure.

FIG. 9 shows a flow scheme of an embodiment similar to that shown in FIG. 7 with the addition of the separation of a stream comprising hydrogen from the CO-enriched exit stream of the CO₂ to CO conversion system. The separated stream comprising hydrogen may be combined with the tail gas recycle. The flow scheme is controlled by one embodiment of the disclosure.

FIG. 10 shows a flow scheme of an embodiment similar to that of FIG. 9 with the addition of a second compressor which operates on the remainder of the CO-enriched exit stream after the stream comprising hydrogen has been separated from the CO-enriched exit stream. The flow scheme is controlled by one embodiment of the disclosure.

FIG. 11 shows a flow scheme of an embodiment similar to that of FIG. 6 with the addition of passing at least a portion of the bioreactor tail gas to a methane conversion unit and passing the effluent of the methane conversion unit to combine back with the bioreactor tail gas. An oxygen source may optionally provide a stream comprising oxygen to the methane conversion unit. A second stream comprising hydrogen from the hydrogen source optionally may be passed directly to the bioreactor. A second stream comprising CO₂ from the CO₂ source optionally may be passed directly to the bioreactor. The flow scheme is controlled by one embodiment of the disclosure.

FIG. 12 shows a flow scheme of an embodiment with greater detail of when the CO₂ to CO conversion system is selected to be a rWGS system. The flow scheme is controlled by one embodiment of the disclosure.

FIG. 13 shows a flow scheme of an embodiment where optionally a portion of hydrogen bypasses the CO₂ to CO conversion system and where optionally a portion of hydrogen is obtained from a second hydrogen source. The flow scheme is controlled by one embodiment of the disclosure.

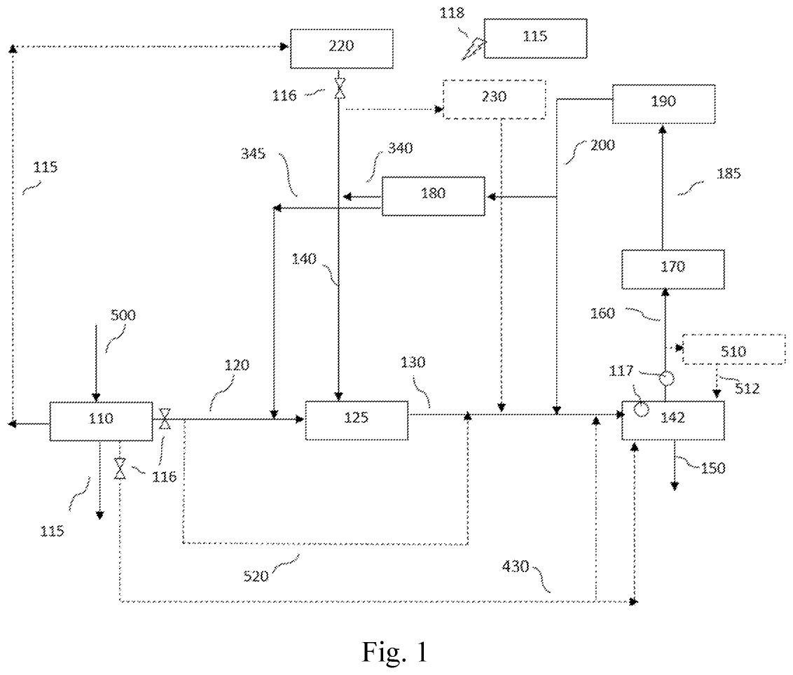

FIGS. 1 through 13 further depict an optional embodiment wherein at least a portion of the input stream to the CO₂ to CO conversion system is bypassed around the CO₂ to CO conversion system instead of passing through the CO₂ to CO conversion system. The figures further show an optional embodiment wherein at least a portion of the tail gas stream is passed through a second CO₂ to CO conversion system and the resulting effluent is passed to the bioreactor. The figures further show an optional embodiment wherein at least a portion of the first gaseous stream comprising H₂ is bypassed around the CO₂ to CO conversion system instead of passing through the CO₂ to CO conversion system. Flow schemes including optional embodiments are controlled by embodiments of the disclosure.

### DETAILED DESCRIPTION

In a gas fermentation process, the integration of a CO₂ generating gas production process such as an industrial process or a syngas process with a CO₂ to CO conversion process, particularly a reverse water gas shift process, provides substantial benefits. The integration allows for the use of CO₂ as a feed stock even when the fermentation process requires a certain amount of CO. Integrating a CO₂ to CO conversion allows for CO₂ in the feed stock or recycle to be converted to CO in the appropriate amount for fermentation

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, or any combination thereof. Examples of specific processing steps within an industrial process include catalyst regeneration, fluid catalyst cracking, and catalyst regeneration. Air separation and direct air capture are other suitable industrial processes. Examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as natural gas, oil, or coal fired boilers or heaters, and gas turbine exhaust. In these embodiments, the substrate and or C1-carbon source may be captured from the industrial process before it is emitted into the atmosphere, using any known method.

The substrate and or C1-carbon source may be synthesis gas known as syngas, which may be obtained from reforming, partial oxidation, plasma, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibres such as in shoes, apparel, and textiles. Municipal solid waste may be simply landfill-type waste and may be sorted or unsorted. Examples of biomass may include lignocellulosic material and microbial biomass. Lignocellulosic material may include agriculture waste and forest waste.

Cl refers to a one-carbon molecule, for example, CO, CO₂, methane (CH₄), or methanol (CH₃OH) and C1-carbon source refers a one carbon-molecule that serves as a partial or sole carbon source for a microorganism of the disclosure. For example, a C1-carbon source may comprise one or more of CO, CO₂, CH₄, CH₃OH, or formic acid (CH₂O₂). A C1-carbon source comprises one or both of CO and CO₂. A substrate is a carbon and or energy source. Typically, the substrate is gaseous and comprises a C1-carbon source, for example, CO, CO₂, and or CH₄. The substrate may further comprise other non-carbon components, such as H₂, N₂, or electrons.

When discussing recycling herein, the description of recycling or passing a stream to a unit is meant to include direct independent introduction of the stream to the unit, or combination of the stream with another input to the unit.

A CO₂ generating gas production process is an industrial process or a syngas process which generates an industrial gas or syngas typically having a significant proportion of CO₂ by volume. Additionally, the industrial gas or syngas may comprise some amount of CO and or CH₄. The CO₂ generating gas production process is intended to include any industrial process or syngas process which generates a CO₂ containing gas as either a desired end product, or as a by-product in the production of one or more desired end products. Exemplary CO₂ generating gas production processes have sources including, ethanol production from a sugar-based ethanol production source, a first generation corn-ethanol production source, a second generation corn-ethanol production source, a sugarcane ethanol production source, a cane sugar ethanol production source, a sugar beet ethanol production source, a molasses ethanol production source, a wheat ethanol production source, a grain based ethanol production source, a starch based ethanol production source, a cellulosic based ethanol production source, a cement production source, a methanol synthesis source, an olefin production source, a steel production source, a ferroalloy production source, a refinery tail gas production source, a post combustion gas production source, a biogas production source, a landfill production source, an ethylene oxide production source, a methanol production source, an ammonia production source, mined CO₂ production source, natural gas processing production source, a gasification source, an organic waste gasification source, direct air capture, or any combination thereof. Some examples in steel and ferroalloy production source include, blast furnace gas, basic oxygen furnace gas, coke oven gas, direct reduction of iron furnace top-gas, electric arc furnace off-gas, and residual gas from smelting iron. Other general examples include flue gas from fired boilers and fired heaters, such as natural gas, oil, or coal fired boilers or heaters, and gas turbine exhaust.

FIG. 1 depicts an integrated system having a flexible production platform and process for the production of at least one fermentation product from a gaseous stream in accordance with one embodiment of the disclosure. The process includes receiving a first gaseous stream comprising hydrogen and a second gaseous stream comprising CO₂ and passing the streams to a CO₂ to CO conversion system. In FIG. 1, CO₂ to CO conversion system 125 is shown as a reverse water gas shift unit. Hydrogen production source 110 generates first gaseous stream comprising hydrogen 120. In one embodiment, hydrogen production source 110 is a water electrolyser. Water stream 500 is introduced to hydrogen production source 110 which may receive power, for example 4.78 kwh/Nm³, from a power source (not shown), to convert water into hydrogen and oxygen according to the following stoichiometric reaction:

H₂O+electricity→2H₂+O₂+heat

Water electrolysis technologies are known, and exemplary processes include alkaline water electrolysis, proton exchange membrane (PEM) electrolysis, and solid oxide electrolysis. Suitable electrolysers include alkaline electrolysers, PEM electrolysers, and solid oxide electrolysers. Oxygen enriched stream 115 comprising oxygen generated as a by-product of water electrolysis may be employed for various purposes. For example, at least a portion of oxygen enriched stream 115 may be introduced to gas production source 220, especially if gas production source 220 is selected to be a syngas production process that includes an oxygen blown gasifier. Such use of oxygen enriched stream 115 reduces the need and associated cost of obtaining oxygen from an external source. The term enriched, as used herein, is meant to describe having a higher concentration after a process step as compared to before the process step.

In specific embodiments, hydrogen production sources 110 may be selected from, hydrocarbon reforming, hydrogen purification, solid biomass gasification, solid waste gasification, coal gasification, hydrocarbon gasification, methane pyrolysis, refinery tail gas production process, a plasma reforming reactor, partial oxidation reactor, or any combinations thereof.

Gas production source 220 generates second gaseous stream comprising CO₂ 140 from direct air capture, a CO₂-generating industrial process, a syngas process, or any combination thereof. First gaseous stream comprising hydrogen 120 and second gaseous stream comprising CO₂ 140 are passed, individually or in combination, to CO₂ to CO conversion system 125 to produce CO enriched exit stream 130. The gas composition of the combination of first gaseous stream comprising hydrogen 120 and second gaseous stream comprising CO₂ 140 comprises an H₂:CO₂ molar ratio of about 3:1 in one embodiment, of about 2.5:1 in another embodiment, and of about 3.5:1 in yet another embodiment, and the H₂:CO molar ratio may be greater than about 5:1. CO₂ to CO conversion system 125 may be at least one unit selected from a reverse water gas shift unit, a thermo-catalytic conversion unit, a partial combustion unit, a reforming unit, or a plasma conversion unit.

In a particular embodiment, CO₂ to CO conversion system 125 is a reverse water gas shift unit. Reverse water gas shift (rWGS) technology is known and is used for producing carbon monoxide from carbon dioxide and hydrogen, with water as a side product. Temperature of the rWGS process is the main driver of the shift. Reverse water gas shift units may comprise a single stage reaction system or two or more reaction stages. The different stages may be conducted at different temperatures and may use different catalysts

In another embodiment, CO₂ to CO conversion system 125 involves thermo-catalytic conversion, which involves disrupting the stable atomic and molecular bonds of CO₂ and other reactants over a catalyst by using thermal energy as the driving force of the reaction to produce CO. Since CO₂ molecules are thermodynamically and chemically stable, if CO₂ is used as a single reactant, large amounts of energy are required. Therefore, often other substances such as hydrogen are used as a co-reactant to make the thermodynamic process easier. Many catalysts are known for the process such as metals and metal oxides as well as nano-sized catalyst metal-organic frameworks. Various carbon materials have been employed as carriers for the catalysts.

In another embodiment, CO₂ to CO conversion system 125 involves partial combustion where oxygen supplies at least a portion of the oxidant requirement for the partial oxidation and the reactants carbon dioxide and water are substantially converted to carbon monoxide and hydrogen.

In still another embodiment, CO₂ to CO conversion system 125 involves plasma conversion which is the combination of plasma with catalysts, also called as plasma-catalysis. Plasma is an ionized gas consisting of electrons, various types of ions, radicals, excited atoms, and molecules, along with neutral ground state molecules. The three most common plasma types for CO₂ to CO conversion include, dielectric barrier discharges (DBDs), microwave (MW) plasmas, and gliding arc (GA) plasmas. Advantages of selecting plasma conversion for CO₂ to CO conversion include (i) high process versatility, allowing different kinds of reactions to be carried out, such as pure CO₂ splitting, as well as CO₂ conversion in presence of a hydrogen source, such as CH₄, H₂ or H₂O; (ii) low investment and operating costs; (iii) no requirement for rare earth metals; (iv) convenient modular setting, as plasma reactors scale up linearly with the plant output; and (v) it can be very easily combined with various kinds of renewable electricity.

The figures are described where CO₂ to CO conversion system 125 is selected to include at least one rWGS unit. The rWGS reaction is the reversible hydrogenation of CO₂ to produce CO and H₂O. Due to its chemical stability, CO₂ it is a relatively unreactive molecule and therefore the reaction to convert it to more reactive CO is energy intensive.

CO₂+H₂↔CO+H₂O ΔH°298 k=+41 kJ mol-1(at standard conditions)

Since the rWGS reaction is endothermic, it is thermodynamically favoured by higher temperatures. Typically, temperature of about 500° C. is desirable to generate significant amount of CO. In embodiments employing higher temperatures, ironbased catalysts are often considered as one of the most successful active metals for higher temperatures, due to its thermal stability and high oxygen mobility. In embodiments employing lower temperatures, copper is often regarded to be successful due to its enhanced adsorption of reaction intermediates. In some other embodiments, rWGS catalysts selections include Fe/Al₂O₃, Fe—Cu/Al₂O₃, Fe—Cs/Al₂O₃, Fe—Cu—Cs/Al₂O₃ or combinations thereof.

CO₂ to CO conversion system 125, employing for example, rWGS technology, produces CO enriched exit stream 130. The H₂:CO molar ratio of the CO enriched exit stream 130 may be greater than about 3:1 in some embodiments. Based on the stoichiometry of ethanol as a product and with CO₂:CO in a molar ratio of 1:1, the H₂:CO:CO₂ molar ratio of the CO enriched exit stream 130 may be about 5:1:1.

In some instances, the rWGS reaction operates at a level such that the H₂:CO molar ratio in the CO enriched exit stream 130 is less than or equal to a predetermined ratio for example about 3:1. Such level of CO may be in excess of the CO level required for gas fermentation. A higher than needed CO conversion from CO₂ to CO conversion system 125 can result in suboptimal performance. Accordingly, CO₂ to CO conversion system 125 size will be designed larger than needed. Such large system is expensive. Therefore, to avoid such large system, at least a portion of first gaseous stream comprising hydrogen is directed to bypass 520 and does not pass to CO₂ to CO conversion system 125. Bypass stream 520 combines with CO enriched exit stream 130. Accordingly, the H₂:CO ratio in line 130 delivered for fermentation may be adjusted to be greater than the predetermined ratio with an optimally sized CO₂ to CO conversion system 125. Similarly, a portion of second gaseous stream comprising CO₂ 140 may be diverted to bypass CO₂ to CO conversion system 125 using second bypass stream 525. In this way, the amount of CO produced may be controlled without overdesigning capacity of CO₂ to CO conversion system 125

If ethanol is not the intended fermentation product, the stoichiometry as discussed above would be different. For example, if 2,3-butanediol (2,3-BDO) was the intended fermentation product, the H₂:CO:CO₂ molar ratio of the CO enriched exit stream 130 may be about 4.5:1:1 based on the stoichiometry of 2,3-BDO and with CO₂:CO in a molar ratio of 1:1.

9H₂+2 CO+2 CO₂→C₄H₁₀O₂+4 H₂O

If acetone was the intended fermentation product, the H₂:CO:CO₂ molar ratio of the CO enriched exit stream 130 may be about 4.33:1:1 based on the stoichiometry of acetone and with CO₂:CO in a molar ratio of 1:1.

6.5 H₂+1.5 CO+1.5 CO₂→C₃H₆O+3.5 H₂O

If acetate was the intended fermentation product, the H₂:CO:CO₂ molar ratio of the CO enriched exit stream 130 may be about 3:1:1 based on the stoichiometry of acetate and with CO₂:CO in a molar ratio of 1:1.

3H₂+1 CO+1 CO₂→C₂H₄O₂+1 H₂O

If isopropyl alcohol was the intended fermentation product, the H₂:CO:CO₂ molar ratio of the CO enriched exit stream 130 may be about 5:1:1 based on the stoichiometry of isopropyl alcohol and with CO₂:CO in a molar ratio of 1:1.

H₂+1.5 CO+1.5 CO₂→C₃H₈O+3.5 H₂O

CO enriched exit stream 130 is passed to bioreactor 142 which contains a culture of one or more Cl fixing bacterium. Bioreactor 142 may be a fermentation system consisting of one or more vessels and or towers or piping arrangements. Examples of bioreactors include continuous stirred tank reactor (CSTR), immobilized cell reactor (ICR), trickle bed reactor (TBR), bubble column, gas lift fermenter, static mixer, circulated loop reactor, membrane reactor, such as hollow fibre membrane bioreactor (HFM BR), or other device suitable for gas-liquid contact. Bioreactor 142 may comprise multiple reactors or stages, either in parallel or in series. Bioreactor 142 may be a production reactor, where most of the fermentation products are produced.

Bioreactor 142 includes a culture of one or more C1-fixing microorganisms that have the ability to produce one or more products from a C1-carbon source. “Cl” refers to a one-carbon molecule, for example, CO or CO₂. “C1-carbon source” refers a one carbon-molecule that serves as a partial or sole carbon source for the microorganism. For example, a C1-carbon source may comprise one or more of CO, CO₂, or CH₂O₂. In some embodiments, the C1-carbon source may comprise one or both of CO and CO₂. Typically, the C1-fixing microorganism is a C1-fixing bacterium. In an embodiment, the microorganism is derived from a C1-fixing microorganism identified in Table 1. The microorganism may be classified based on functional characteristics. For example, the microorganism may be derived from a C1-fixing microorganism, an anaerobe, an acetogen, an ethanologen, and or a carboxydotroph. Table 1 provides a representative list of microorganisms and identifies their functional characteristics.

TABLE 1

| Table 1 | C1-fixing | Anaerobe | Acetogen | Ethanologen | Autotroph | Carboxydotroph | Methanotroph |
|---|---|---|---|---|---|---|---|
| Acetobacterium woodii | + | + | + | +/− ¹ | − | +/− ² | − |
| Alkalibaculum bacchii | + | + | + | + | + | + | − |
| Blautia product | + | + | + | − | + | + | − |
| Butyribacterium methylotrophicum | + | + | + | + | + | + | − |
| Clostridium aceticum | + | + | + | − | + | + | − |
| Clostridium autoethanogenum | + | + | + | + | + | + | − |
| Clostridium carboxidivorans | + | + | + | + | + | + | − |
| Clostridium coskatii | + | + | + | + | + | + | − |
| Clostridium drakei | + | + | + | − | + | + | − |
| Clostridium formicoaceticum | + | + | + | − | + | + | − |
| Clostridium ljungdahlii | + | + | + | + | + | + | − |
| Clostridium magnum | + | + | + | − | + | +/− ³ | − |
| Clostridium ragsdalei | + | + | + | + | + | + | − |
| Clostridium scatologenes | + | + | + | − | + | + | − |
| Eubacterium limosum | + | + | + | − | + | + | − |
| Moorella thermautotrophica | + | + | + | + | + | + | − |
| Moorella thermoacetica (formerly | + | + | + | − ⁴ | + | + | − |
| Clostridium thermoaceticum) | | | | | | | |
| Oxobacter pfennigii | + | + | + | − | + | + | − |
| Sporomusa ovata | + | + | + | − | + | +/− ⁵ | − |
| Sporomusa silvacetica | + | + | + | − | + | +/− ⁶ | − |
| Sporomusa sphaeroides | + | + | + | − | + | +/− ⁷ | − |
| Thermoanaerobacter kivui | + | + | + | − | + | − | − |

An “anaerobe” is a microorganism that does not require oxygen for growth. An anaerobe may react negatively or even die if oxygen is present above a certain threshold. Typically, the microorganism is an anaerobe (i.e., is anaerobic). In one embodiment, the microorganism is or is derived from an anaerobe identified in Table 1.

An “acetogen” is a microorganism that produces or is capable of producing acetate (or acetic acid) as a product of anaerobic respiration. Typically, acetogens are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). Acetogens use the acetyl-CoA pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3rd edition, p. 354, New York, N.Y., 2006). All naturally occurring acetogens are C1-fixing, anaerobic, autotrophic, and non-methanotrophic. In one embodiment, the microorganism is an acetogen. In one embodiment, the microorganism is or is derived from an acetogen identified in Table 1.

More broadly, the microorganism may be derived from any genus or species identified in Table 1. For example, the microorganism may be a member of the genus Clostridium. In one embodiment, the microorganism is obtained from the cluster of Clostridia comprising the species Clostridium autoethanogenum, Clostridium ljungdahlii, and Clostridium ragsdalei. These species were first reported and characterized by Abrini, Arch Microbiol, 161: 345-351, 1994 (Clostridium autoethanogenum), Tanner, Int J System Bacteriol, 43: 232-236, 1993 (Clostridium ljungdahlii), and Huhnke, WO 2008/028055 (Clostridium ragsdalei). The microorganism may also be derived from an isolate or mutant of Clostridium autoethanogenum, Clostridium ljungdahlii, or Clostridium ragsdalei. Isolates and mutants of Clostridium autoethanogenum include JA1-1 (DSM10061) (Abrini, Arch Microbiol, 161: 345-351, 1994), LBS1560 (DSM19630) (WO 2009/064200), and LZ1561 (DSM23693). Isolates and mutants of Clostridium ljungdahlii include ATCC 49587 (Tanner, Int J Syst Bacteriol, 43: 232-236, 1993), PETCT (DSM13528, ATCC 55383), ERI-2 (ATCC 55380) (U.S. Pat. No. 5,593,886), C-01 (ATCC 55988) (U.S. Pat. No. 6,368,819), 0-52 (ATCC 55989) (U.S. Pat. No. 6,368,819), and OTA-1 (Tirado-Acevedo, Production of bioethanol from synthesis gas using Clostridium ljungdahlii, PhD thesis, North Carolina State University, 2010). Isolates and mutants of Clostridium ragsdalei include PI 1 (ATCC BAA-622, ATCC PTA-7826) (WO 2008/028055).

The microorganism of the disclosure may be cultured to produce one or more products. For instance, Clostridium autoethanogenum produces or can be engineered to produce ethanol (WO 2007/117157), acetate (WO 2007/117157), butanol (WO 2008/115080 and WO 2012/053905), butyrate (WO 2008/115080), 2,3-butanediol (WO 2009/151342), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), isoprene (WO 2013/180584), fatty acids (WO 2013/191567), 2-butanol (WO 2013/185123), 1,2-propanediol (WO 2014/0369152), and 1-propanol (WO 2014/0369152). In addition to one or more target products, the microorganism of the disclosure may also produce ethanol, acetate, and or 2,3-butanediol. In certain embodiments, microbial biomass itself may be considered a product.

The culture is generally maintained in an aqueous culture medium that contains nutrients, vitamins, and or minerals sufficient to permit growth of the microorganism. The aqueous culture medium may be an anaerobic microbial growth medium, such as a minimal anaerobic microbial growth medium. Suitable media are well known in the art.

The culture and or fermentation may be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase and thus provides an advantage. Also, since a given gas conversion rate is in part a function of the substrate retention time, the conversion rate dictates the required volume of a bioreactor. The use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. In specific embodiments the fermentation is operated at a pressure higher than atmospheric pressure.

Target products may be separated the fermentation broth using any method or combination of methods known in the art, including, for example, fractional distillation, evaporation, pervaporation, gas stripping, phase separation, extractive separation, including for example, liquid-liquid extraction. In certain embodiments, target products are recovered from the fermentation broth by continuously removing a portion of the broth from the bioreactor, first separating microbial cells from the broth and then separating the target product from the aqueous remainder. Alcohols and or acetone may be recovered, for example, by distillation. Acids may be recovered, for example, by adsorption on activated charcoal. Separated microbial biomass may be recycled to the bioreactor. The solution remaining after the target products have been removed may also be recycled to the bioreactor. Additional nutrients may be added to the recycled solution to replenish the medium before it is returned to the bioreactor.

CO enriched exit stream 130 is introduced to bioreactor 142 and is fermented to produce tail gas stream 160 and fermentation product stream 150 that may comprise any of the products described above. The term tail gas refers to gasses and vapors ordinarily released into the atmosphere from an industrial process after all reactor and treatment has taken place. Tail gas stream 160 is ultimately recycled combine with second gaseous stream comprising CO₂ 140 for introduction to CO₂ to CO conversion system 125. Tail gas stream 160 may include some amount of CO₂ produced during the fermentation, for example by the reaction:

6CO+3H₂O→C₂H₅OH+4CO₂(ΔG°=−224.90 kJ/mol ethanol)

Recycling CO₂ present in tail gas stream 160 from bioreactor 142 to CO₂ to CO conversion system 125 increases the efficiency of the carbon capture of the overall process. Tail gas stream 160 depleted in CO may comprise less than about 5 mol % CO. The H₂:CO₂ molar ratio of tail gas stream 160 in some embodiments is equal to or less than about 3:1.

Tail gas stream 160 may include various constituents that are best removed before further processing. In these instances, tail gas stream 160 is treated to remove one or more constituents and produce a desulfurized and or acid gas treated tail gas stream 340 which may be combined with second gaseous stream comprising CO₂ 140. The one or more constituents which may be removed from tail gas stream 160 may include, sulfur-containing compounds, including, without limitation, hydrogen sulfide (H₂S), carbon disulfide, and or sulfur dioxide, aromatic compounds, alkynes, alkenes, alkanes, olefins, nitrogen compounds, phosphorous-containing compounds, particulate matter, solids, oxygen, oxygenates, halogenated compounds, silicon-containing compounds, carbonyls, metals, alcohols, esters, ketones, peroxides, aldehydes, ethers, tars, methanethiol, ammonia, diethylamine, triethylamine, acetic acid, methanol, ethanol, propanol, butanol and higher alcohols, naphthalene, or combinations thereof. These constituents may be removed by conventional removal modules known in the art, such as hydrolysis module, acid gas removal module, deoxygenation module, catalytic hydrogenation module, particulate removal module, chloride removal module, tar removal module, and or hydrogen cyanide removal module, and combinations thereof. In particular instances, at least one constituent removed from the tail gas stream include sulfur-containing compounds such as hydrogen sulfide that may be produced, introduced, and or concentrated by the fermentation process. Hydrogen sulfide may be a catalyst inhibitor in the CO₂ to CO system 125 employing rWGS technology and catalysts.

Tail gas stream 160 is passed through gas component removal unit 170. Gas component removal unit 170 removes constituents other than sulfur-containing compounds or acid gas components. In some embodiments, the component removed is water. Because the water gas shift reaction produces water, it is advantageous to limit the amount of water fed to the water gas shift reactors. Removing water allows for better water balance across the overall process. In some embodiments the component removed is hydrocarbons. Gas component removal unit 170 may include multiple submodules in order to remove multiple constituents other than sulfur-containing compounds. In some embodiments, liquid scrubbers are used to remove ethanol including other soluble components and higher alcohols. In these embodiments, gas component removal unit 170 may be operating to capture and recover fermentation product included in tail gas stream 160. Volatile organic compounds may also be removed in gas component removal unit 170. Other components that may be removed in gas component removal unit 170 include, for example, mono nitrogenous species such as hydrogen cyanide (HCN), ammonia (NH₃), nitrogen oxide (NOₓ) and other known enzyme inhibiting gases such as acetylene (C₂H₂), ethylene (C₂H₄), ethane (C₂H₆), benzene, toluene, ethyl benzene, xylene, (BTEX), and or oxygen (O₂).

Resulting treated tail gas stream 185 is passed to a first compressor 190 to generate compressed treated gas stream 200 which is passed to gas desulfurization/acid gas removal unit 180. In some embodiments, compressor 190 may be positioned upstream of gas component removal unit 170 between bioreactor 142 and gas component removal unit 170 to compress tail gas stream 160 prior to passing to gas component removal unit 170. Generally, compressor 190 is operated at a pressure from about 3 Barg to about 10 Barg. Compressed treated tail gas stream 200 is passed to gas desulfurization/acid gas removal unit 180 to produce desulfurized and or acid gas treated tail gas stream 340. Gas desulfurization/acid gas removal unit 180. Sulfur-containing compounds and or acid gases are removed as they act as inhibitors in CO₂ to CO conversion system 125 using rWGS technology by poisoning rWGS catalysts. Many commercial desulfurization technologies cannot efficiently remove sulfur in the form of COS but are better able to handle sulfur in the form of hydrogen sulfide. In one embodiment, gas desulfurization/acid gas removal unit 180 operates to convert compounds such as carbonyl sulfide COS to hydrogen sulfide H₂S by hydrolysis according to the following reaction:

COS+H₂O ↔H₂S+CO₂

The hydrolysis may be accomplished by a metal oxide catalyst or an alumina catalyst to perform the conversion of COS to H₂S. In some embodiments, two or more desulfurization operations may be employed, such as an iron sponge followed by a metal oxide catalyst. In certain other embodiments, gas desulfurization/acid gas removal unit 180 may employ a zinc oxide (ZnO) catalyst to remove hydrogen sulfide. In other embodiments, pressure swing adsorption (PSA) is utilized to remove acid gas by adsorption through suitable adsorbents in fixed beds contained in vessels under high pressure. In yet other embodiments, caustic scrubbing is used for gas desulfurization. Caustic scrubbing may include passing compressed treated tail gas stream 200 through a caustic solution such as NaOH to remove sulfur-containing compounds. Removal of hydrogen sulfide by caustic scrubbing may be represented as follows:

H₂S(g)+NaOH(aq)→NaHS(aq)+H₂O(l)

NaHS(aq)+NaOH(aq)→Na₂S(aq)+H₂O

Desulfurized and or acid gas treated tail gas stream 340 exiting from gas desulfurization/acid gas removal unit 180 may be combined with second gaseous stream comprising CO₂ 140 and recycled to CO₂ to CO conversion system 125. Alternatively, instead of desulfurized and or acid gas treated tail gas stream 340 being passed to combine with the second gaseous stream comprising CO₂ 140, alternative desulfurized and or acid gas treated tail gas stream 345 is combined with first gaseous stream comprising hydrogen 120.

A portion of compressed treated tail gas stream 200 may combined with CO enriched exit stream 130 and passed to bioreactor 142 instead of being passed to gas desulfurization/acid gas removal unit 180. Such recycling benefits microorganism growth because the microorganisms consume sulfur to produce amino acids, for example, methionine and cysteine. Consequentially, sulfur dosing requirements to bioreactor 142 are reduced due to sulfur recycling as a portion of compressed treated tail gas stream 200.

In an optional embodiment where gas production source 220 involves production of biogas, a portion of second gaseous stream comprising CO₂ 140 is passed to optional biogas reformer 230. Biogas refers to a gas produced by the anaerobic digestion of organic matter such as manure, sewage sludge, municipal solid waste, biodegradable waste, or any other biodegradable feedstock. Biogas is comprised primarily of methane and carbon dioxide. Generally, in a biogas reformer combined CO₂ and steam reforming of methane is carried out to produce a syngas stream.

CH₄+CO₂↔2CO+2H₂ΔH°=247 kJ/mol

CH₄+H₂O↔CO+3H₂ΔH°=206 kJ/mol

With respect to FIG. 1, biogas reformer effluent stream 240 comprising CO and H₂ produced in biogas reformer 230 is combined with CO enriched exit stream 130 and may operate to improve the H₂:CO ratio for many fermentation processes.

In one embodiment, at least a portion tail gas stream 160 is passed through optional second CO₂ to CO conversion system 510 which may be a reverse water gas shift unit, a thermo-catalytic conversion unit, a partial combustion unit, a plasma conversion unit, a gasification unit, or a reforming unit. Tail gas stream 160 is lean in CO but may have residual H₂ and CO₂. Passing at least a portion of tail gas stream 160 through optional second CO₂ to CO conversion system 510 and recycling second CO₂ to CO conversion system effluent 512 to bioreactor 142 may lower the H₂:CO ratio in bioreactor 142. Such lowering of the H₂:CO ratio in bioreactor 142 may benefit product selectivity and increased or faster microbial growth. Note that second CO₂ to CO conversion system effluent 512 may be recycled to combine with stream 130 instead of being independently passed to bioreactor 142 (not shown).

In one embodiment, optional additional stream comprising hydrogen 430 generated from hydrogen production source 110 is passed to bioreactor 142 or to CO enriched exit stream 130, thus bypassing CO₂ to CO conversion system 125. Additional stream comprising hydrogen 430 may be passed to without intervening processing units. Microbial fermentation of CO in the presence of H₂ can lead to substantially complete carbon transfer into a product such as an alcohol, but, in the absence of sufficient H₂, only a portion of available CO is converted into product, while another portion is converted to CO₂ as in the following equation: 6CO+3H₂O→C₂H₅OH+4CO₂. Therefore, providing sufficient hydrogen to bioreactor 142 may be beneficial in some embodiments. Employing the bypass of the additional stream comprising hydrogen 430 being passed to bioreactor 142 or to CO enriched exit stream 130 without passing through CO₂ to CO conversion system 125 allow for control the of amount the hydrogen being directed to the units at different times of overall process run. For example, during start-up less hydrogen maybe needed in the bioreactor including any inoculator thereby benefitting from CO-rich feeds at start-up. However, toward the end of a run, less CO may be required in the bioreactor and a greater relative amount of H₂ may be employed. This may be particularly beneficial at turndown, or inoculation stage (where main bioreactors receive less CO than a inoculation bioreactors), or when employing a buffer tank. The bypass enables control to vary the H₂:CO ratio of the feed to CO₂ to CO conversion system 125, to bioreactor 142, or both. The bypass also allows for control to vary the H₂:C (hydrogen:carbon) to CO₂ to CO conversion system 125, bioreactor 142, or both.

Providing a CO-rich environment in bioreactor 142 through use of CO₂ to CO conversion system 125 and recycling CO₂ from bioreactor 142 to CO₂ to CO conversion system 125 may benefit product selectivity for those products having improved productivity in gas environment with a higher proportion of CO. One such example is the production of ethanol. Another benefit is that microbial growth of particular microorganisms having the Wood-Ljungdahl pathway may increase, because when those microbes consume higher concentrations of CO, the biological water gas shift in the Wood-Ljungdahl pathway is improved.

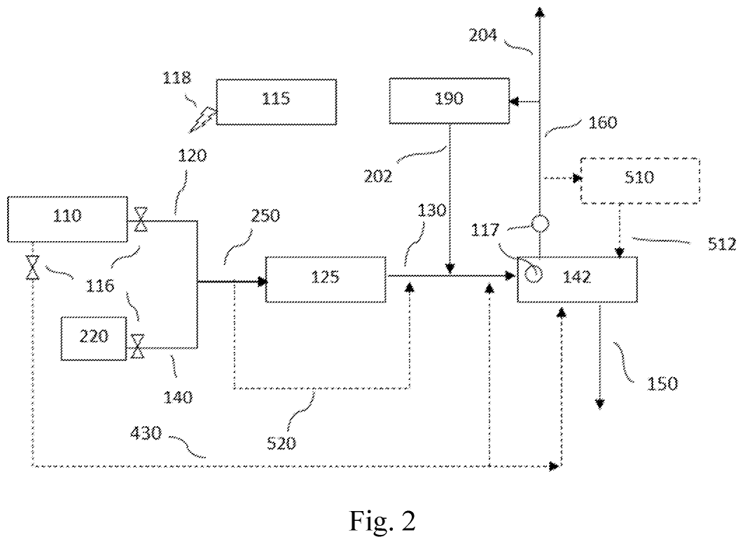

FIG. 2 shows an integrated system for the production of at least one fermentation product from a gaseous stream in accordance with another embodiment of the disclosure. Hydrogen production source 110 generates first gaseous stream comprising hydrogen 120, Gas production source 220, which may be direct air capture or a CO₂ generating industrial process, generates second gaseous stream comprising CO₂ 140. First gaseous stream comprising hydrogen 120 and second gaseous stream comprising CO₂ 140 are combined to form combined feed stream 250 and passed to CO₂ to CO conversion system 125. The gas composition in combined feed stream 250 comprises a H₂:CO₂ molar ratio of about 3:1 in one embodiment of about 2.5:1 in another embodiment, of about 3.5:1 in yet another embodiment, and greater than about 5:1 in still another embodiment.

In one embodiment, CO₂ to CO conversion system 125 employs rWGS technology. In CO₂ to CO conversion system 125, CO₂ is reacted to produce CO enriched exit stream 130. Molar ratios of components in stream are as discussed in FIG. 1. As shown in FIG. 2, in an embodiment, at least a portion of feed stream 250 is optionally diverted around CO₂ to CO conversion system 125 in bypass stream 520. Bypass stream 520 combines with CO enriched exit stream 130. The benefits of bypass stream 520 is as described in FIG. 1. CO enriched exit stream 130 is passed to bioreactor 142 having a culture of one or more Cl fixing microorganisms. The culture is fermented to produce one or more fermentation products 150 and tail gas stream 160. Tail gas stream 160 is depleted in CO may comprise less than about 5 mol % CO. The H₂:CO₂ molar ratio of tail gas stream 160 in some embodiments, is less than or equal to about 3:1.

Tail gas stream 160 is passed to first compressor 190 to produce compressed tail gas stream 202. Compressed tail gas stream 202 is recycled to combine with CO enriched exit stream 130. Optionally, a small first purge stream 204 of tail gas stream 160 or small second purge stream 206 of compressed tail gas stream 202 may be removed to control nitrogen, methane, argon, helium, or other inert component accumulation.

As in FIG. 1, in one embodiment, at least a portion tail gas stream 160 is passed through optional second CO₂ to CO conversion system 510 and second CO₂ to CO conversion system effluent 512 is recycled to bioreactor 142 or to CO enriched exit stream. Also as in FIG. 1, that second CO₂ to CO conversion system effluent 512 may be recycled to combine with stream 130 instead of being independently passed to bioreactor 142 (not shown).

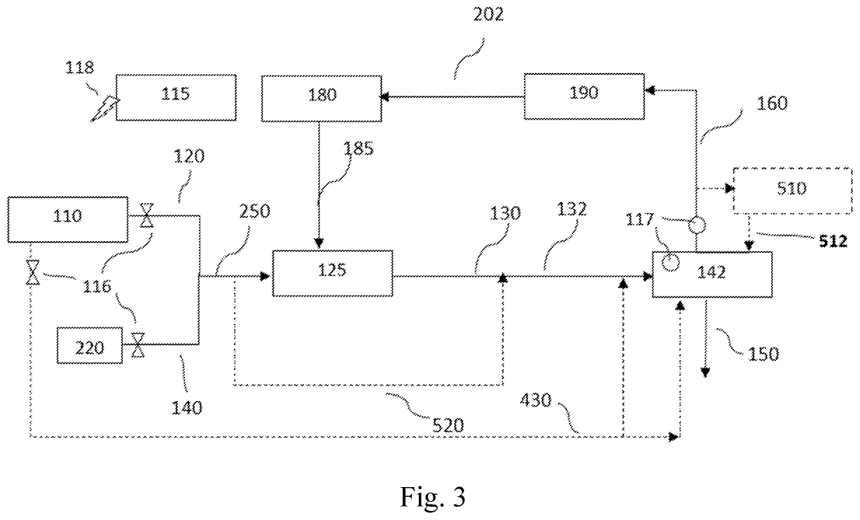

FIG. 3 shows another embodiment similar to FIG. 2, except that compressed tail gas stream 202 is passed to gas desulfurization/acid gas removal unit 180 and resulting desulfurized and or acid gas treated tail gas stream 340 is passed to CO₂ to CO conversion system 125. The gas composition in combined feed stream 250, in CO enriched exit stream 130 and in tail gas stream 160 is as described in FIGS. 1 and 2. Optional bypass related embodiments are as described in FIG. 2.

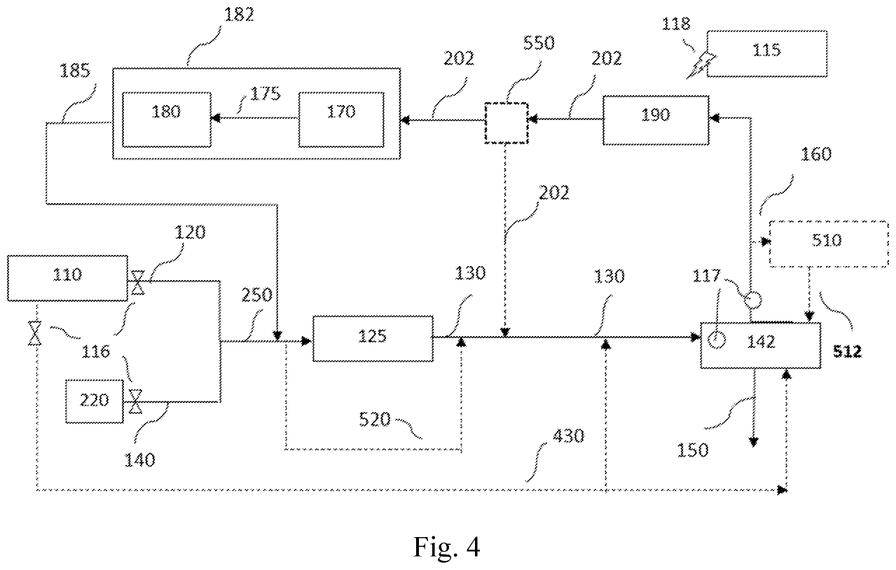

FIG. 4 shows another embodiment similar to FIGS. 2 and 3. Tail gas stream 160 is passed to first compressor 190 and resulting compressed tail gas stream 202 is passed to optional control valve 550. Optional control valve 550 is used to control the relative portions of compressed tail gas stream 202 that is directed to gas treatment zone 182 or to combine with CO enriched exit stream 130. Gas treatment zone 182 is shown as including gas component removal unit 170 and gas desulfurization/acid gas removal unit 180. However, both units may not be required in all embodiments, and gas treatment zone 182 may contain only one of gas component removal unit 170 or gas desulfurization/acid gas removal unit 180. Furthermore, the units in gas treatment zone 182 may be in any order. Treated tail gas stream 185 generated from gas treatment zone 182 is added to combined feed stream 250 and passed to CO₂ to CO conversion system 125. Optional control valve 550 may be adjusted to divide compressed tail gas stream 202 at different proportions based on the phase of fermentation occurring at the time. For example, during start-up fermentation phase, increased CO demands in bioreactor 142 may be met by adjusting control valve 550 to flow more of compressed tail gas stream 202 to combine with CO enriched exit stream 130 than to gas treatment zone 182. On the other hand, as fermentation in bioreactor 142 transitions to stable phase, decreased CO demands of bioreactor 142 may be met by adjusting control valve 550 to flow less of compressed tail gas stream 202 to combine with CO enriched exit stream 130 than to gas treatment zone 182. In other instances, if H₂ utilization during fermentation is low, for example, less than 70%, control valve 550 may be adjusted to flow more of compressed tail gas stream 202 to combine with CO enriched exit stream 130 than to gas treatment zone 182. As shown, control valve 550 is used to accomplish dynamic control of the H₂:CO ratio provided to bioreactor 142 based on the CO and or the H₂ requirements during fermentation. Gas compositions are described with respect to FIG. 2 and FIG. 3 and optional bypass embodiments are as described in FIG. 2.

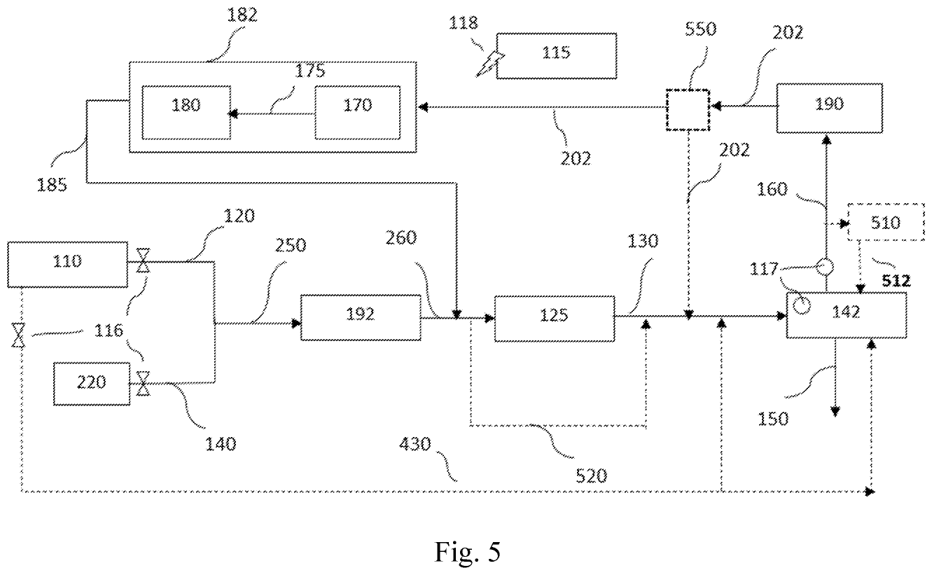

FIG. 5 is similar to FIG. 4 with the addition of second compressor 192. Combined feed stream 250 is passed to second compressor 192 to produce compressed combined feed stream 260. Gas composition of combined feed stream 260 is as discussed above. Compressed combined feed stream 260 is combined with treated tail gas stream 185 and passed to CO₂ to CO conversion system 125 to generate CO enriched exit stream 130. Gas compositions of CO enriched exit stream 120 and tail gas stream 160 are described above. Optional control valve 550, and bypass embodiments are as discussed above.

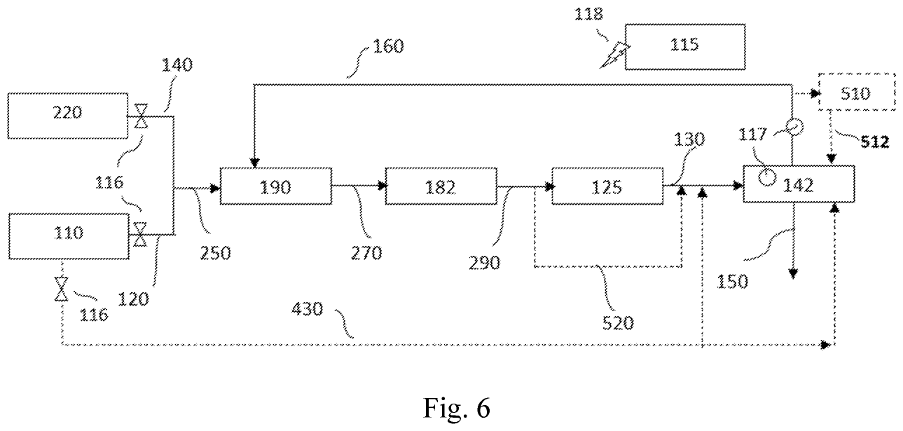

FIG. 6, shows an embodiment wherein both combined feed stream 250 and tail gas stream 160 are passed to first compressor 190. First compressor 192 provides compressed stream 270 which is passed to gas treatment zone 182. Gas treatment zone is as described above. It is understood that some gas treatment modules may be added or removed to gas treatment zone 182 based on actual gas composition. For example, in some embodiments, compressed stream 270 may include acetylene (C₂H₂) which may act as a microbe inhibitor in the fermentation. To remove acetylene, a catalytic hydrogenation module may be included in gas treatment zone 182. Catalytic hydrogenation involves adding hydrogen in presence of hydrogenation catalysts such as those comprising nickel, palladium, platinum. The choice of hydrogenation catalyst depends upon the specific gas composition and operating conditions of the system. In particular embodiments, palladium on alumina (Pd/Al₂O₃) is used as the catalyst. An example of such a catalyst is the BASF™ R 0-20/47. In other embodiments, the gas composition of compressed stream 270 may include benzene, ethyl benzene, toluene, and xylene (BETX) which may inhibit fermentation. Therefore a BETX removal module may be added to gas treatment zone 182. An exemplary BETX removal module may involve adsorption of BETX components using one or more beds of activated carbon. Another exemplary BTEX removal modules involves vent gas incineration which is a thermal oxidation process in which the BTEX components are combusted at temperatures in excess of about 650° C. Treated stream 290 is passed to CO₂ to CO conversion system 125. Gas compositions of various streams are presented above. Bypass embodiments are as described above.

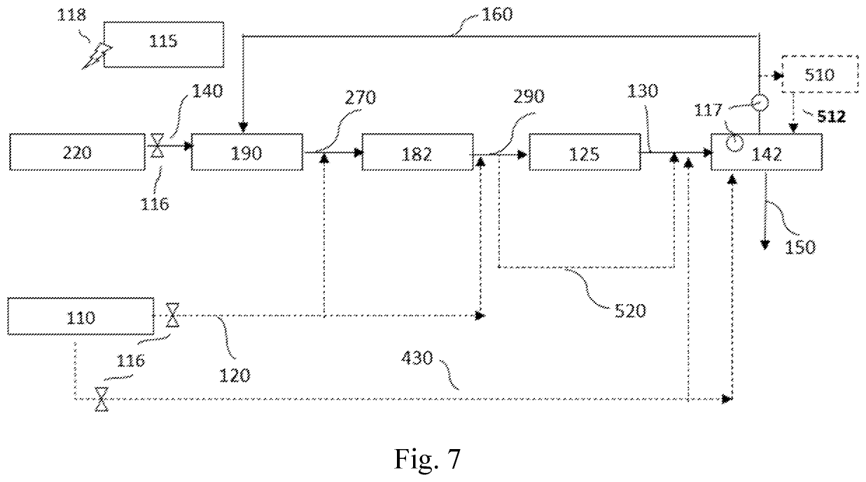

FIG. 7 is similar to FIG. 6, except that first gaseous stream comprising hydrogen 120 may be already pressurized by virtue of hydrogen source 110 and therefore does not need to be passed to first compressor 190. First gaseous stream comprising hydrogen 120 may be combined with second gaseous stream comprising CO₂140 before, after, or both before and after gas treatment zone 182 without being passed through first compressor 190. The gas composition in the gas stream 290 before introducing to the CO₂ to CO conversion system 125 comprises H₂:CO₂ molar ratio of about 3:1 in one embodiment, about 2.5:1 in another embodiment, about 3.5:1 in yet another embodiment, and greater than about 5:1 in yet another embodiment. Gas composition in the CO enriched exit stream 130 and in the tail gas stream 160 is as described above.

FIG. 7 also shows the embodiment where, regardless of the pressure provided by hydrogen source 110, first gaseous stream comprising hydrogen 120 is optional and may not be employed in favor of instead, employing stream comprising hydrogen 430 generated from hydrogen production source 110 which does not pass through CO₂ to CO conversion system 125. Additional stream comprising hydrogen 430 may be passed to bioreactor 142 or to combine with CO enriched exit stream 130. If necessary, stream comprising hydrogen 430 generated from hydrogen production source 110 may be compressed to a target pressure. Keeping the supply of CO₂ separate from the supply of H₂ allow for increased control the of amount the hydrogen being directed to bioreactor 142 at different times of overall process run. For example, during start-up less hydrogen maybe needed in the bioreactor including any inoculator thereby benefitting from CO-rich feeds at start-up. However, toward the end of a run, less CO may be required in the bioreactor and a greater relative amount of H₂ may be employed. This may be particularly beneficial at turndown, or inoculation stage (where main bioreactors receive less CO than a inoculation bioreactors), or when employing a buffer tank. The bypass enables control to vary the H₂:CO ratio of the feed to CO₂ to CO conversion system 125, to bioreactor 142, or both. One target H₂:CO:CO₂ ratio for the bio reactor may be 1:3:1.

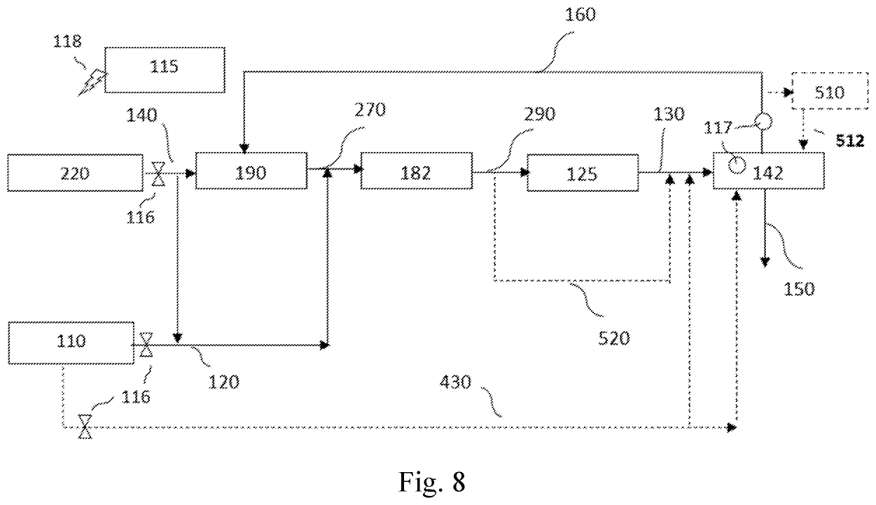

In FIG. 8, a portion of second gaseous stream comprising CO₂ 140 is passed to first compressor 190 while another portion of second gaseous stream comprising CO₂ 140 is combined with first gaseous stream comprising hydrogen 120 and passed to gas treatment zone 182, thereby bypassing first compressor 190. Some gas production sources 220 may provide oxygen as a component of the second gaseous stream comprising CO₂ 140. However, for some microorganisms, oxygen may be a microbe inhibitor and oxygen content in the second gaseous stream comprising CO₂ 140 may need to be reduced to acceptable levels. In these situations, gas treatment zone 182 may further comprise a deoxygenation module. The deoxygenation module may employ a catalytic process whereby oxygen is reduced to either CO₂ or water. In particular embodiments, the catalyst used in the deoxygenation module includes copper. An example of a such a catalyst is BASF PURISTAR™ R 3.15 or BASF CU 0226S. The deoxygenation process is exothermic, and the heat produced may be used within the overall process, such as to preheat the gas prior to an endothermic reaction in CO₂ to CO conversion system 125 involving rWGS technology. The gas composition of various streams are described above. Bypass embodiments are described above.

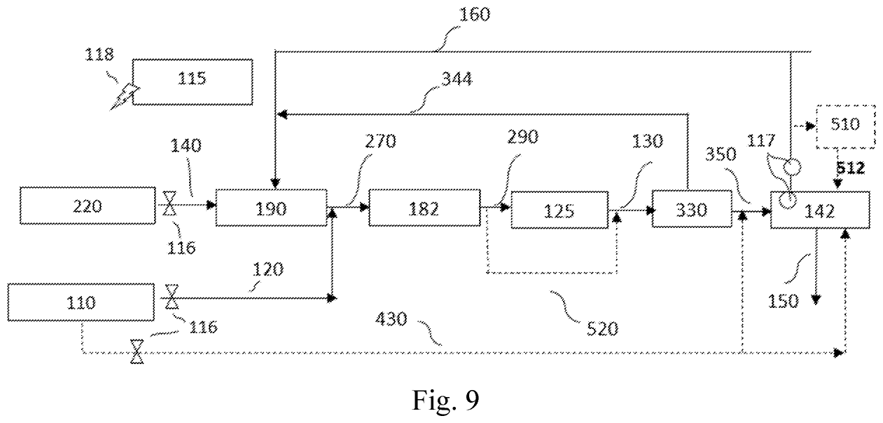

FIG. 9 shows an embodiment wherein CO enriched exit stream 130 from CO₂ to CO conversion system 125 is passed through hydrogen separation unit 330 prior to being passed to bioreactor 142. Hydrogen separation unit 330 may involve membrane separation technology or pressure swing adsorption technology. Separating hydrogen from CO enriched exit stream 130 increases the amount of CO in the H₂:CO ratio of hydrogen separation unit effluent 350 which is passed to bioreactor 142. Separated hydrogen stream 344 generated in hydrogen separation unit 330 is recycled to first compressor 190 separately (not shown) or combined with tail gas stream 160 also being recycled to first compressor 190. FIG. 9 shows an embodiment wherein the first gaseous stream comprising hydrogen 120 is already at sufficient pressure and therefore bypasses first compressor 190 to combine with compressed stream 270 prior to gas treatment zone 182. If first gaseous stream comprising hydrogen 120 was not already at pressure, at least a portion of first gaseous stream comprising hydrogen 120 may be passed through first compressor 190. The gas composition of treated stream 290 before introducing to CO₂ to CO conversion system 125 comprises H₂:CO₂ molar ratio of about 3:1 in one embodiment, about 2.5:1 in another embodiment, about 3.5:1 in yet another embodiment and greater than about 5:1 in still another embodiment. The H₂:CO gas composition in the CO enriched exit stream 130 is as described above. In one embodiment, gas composition in hydrogen separation zone effluent 350 comprises a H₂:CO molar ratio greater than about 1:1 but not exceeding about 5:1, and a H₂:CO:CO₂ molar ratio of about 5:1:1 where ethanol is the product as described above, and further as described above for other products. Gas composition of the tail gas stream 160 is as described above. Bypass embodiments are generally as described above.

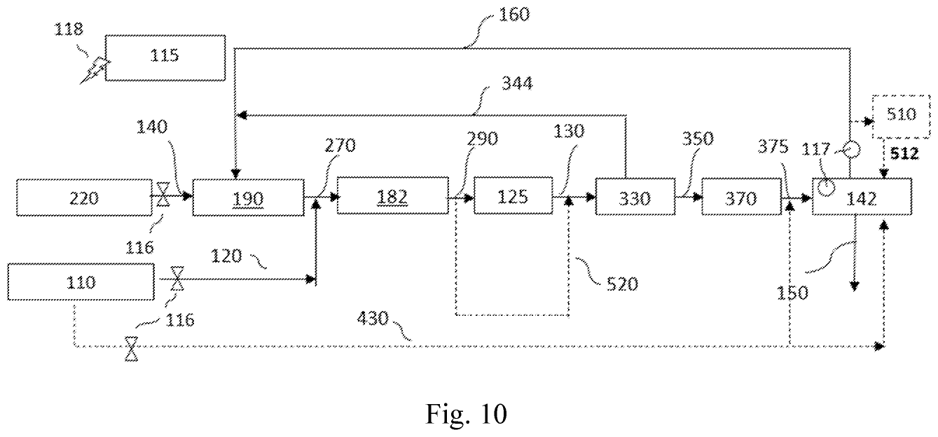

FIG. 10 is similar to FIG. 9 with an added hydrogen separation unit effluent compressor 370. When hydrogen separation unit 330 employs pressure swing adsorption, hydrogen separation unit effluent 350 is often below the pressure needed for bioreactor 142. Hydrogen separation unit effluent compressor 370 provided further compression of hydrogen separation unit effluent to achieve the necessary pressure for introduction into bioreactor 142. The gas composition of treated stream 290 before introducing to the CO₂ to CO conversion system 125 and in CO enriched exit stream 130 are as discussed above. Gas composition of hydrogen separation unit effluent before introducing to hydrogen effluent zone effluent compressor 370 comprises a H₂:CO molar ratio greater than about 1:1 but not exceeding about 5:1, and the H₂:CO:CO₂ molar ratio of the gas stream 365 may be about 5:1:1 for ethanol as the product as described above, and further as described above for other products. Gas composition in the tail gas stream 160 is as described above. Bypass embodiments are as described above.

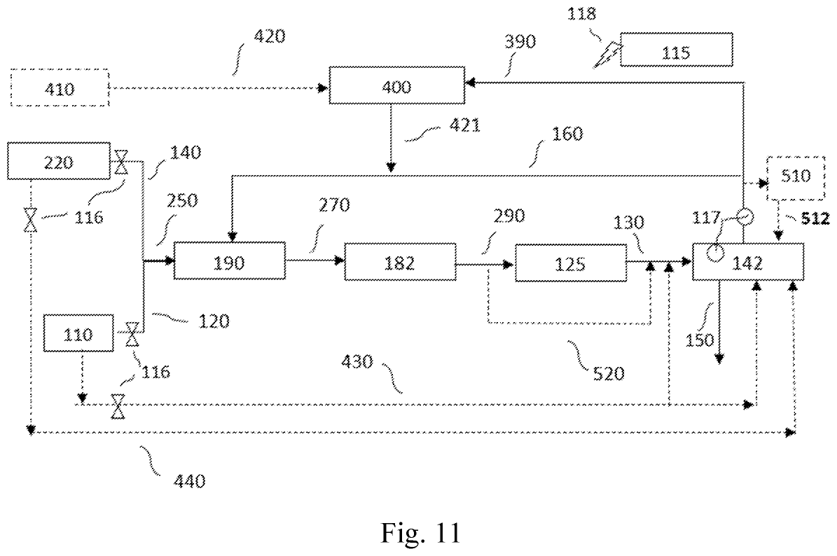

FIG. 11 is similar to FIG. 6, except that CO enriched exit stream 130 from the CO₂ to CO conversion system 125 further includes methane either from hydrogen source 110 or as a by-product of CO₂ to CO conversion system 125 involving rWGS technology. Over time, methane from either or both of these sources might accumulate in bioreactor tail gas stream 160. As the methane concentration of bioreactor tail gas stream 160 increases to a threshold limit of, for example, over 10 mol %, and perhaps more than 50 mol %, at least a portion of tail gas stream 160 is passed as tail gas purge 390 to methane conversion unit 400. Optional oxygen source 410 may provide optional stream comprising oxygen 420 to methane conversion unit 400. In some embodiments, oxygen source 410 for the methane conversion unit 400 may be a water electrolyzer whereby oxygen is a by-product. Methane conversion unit 400 produces at least CO₂ by oxidation of methane according to the reaction CH₄+2O₂→CO₂+2H₂O and generates methane conversion effluent stream 421 comprising at least CO₂ and likely additionally comprising CO and H₂, which may be combined with tail gas stream 160 and passed to first compressor 190. Methane conversion unit 400 may be a methane reforming unit, a methane steam reforming unit, a partial oxidation unit, an auto thermal reforming unit, an oxidation unit, a combustion unit, a biogas reforming unit, or a gasification unit. When methane conversion unit 400 involves steam reforming of methane represented by following equation:

CH₄+H₂O(steam)→CO+3H₂ (endothermic)

Stream comprising oxygen 420 may also be combusted in burners of heaters to create steam or heat the methane conversion unit. Methane conversion unit may involve autothermal reforming (ATR) which uses oxygen or carbon dioxide as reactants with methane to form syngas. The reaction may take place in a single reactor where the methane is partially oxidized. The reactions can be described in the following equations:

CH₄+O₂+CO₂→3 H₂+3 CO+H₂O(using CO₂)

CH₄+O₂+2 H₂O→10H₂+4 CO (using steam)

The gas composition of treated stream 290 and CO enriched exit stream 130 are as described above. The gas composition in the tail gas stream 160 or tail gas purge 390 typically comprises less than about 5 mol % CO. The H₂:CO₂ molar ratio of the tail gas stream 160 or tail gas purge 390 in some embodiments is equal to or less than about 3:1 and the accumulated methane is greater than about 5 mol %. Bypass embodiments are as discussed previously.

In one embodiment, as discussed above, optional additional stream comprising hydrogen 430 generated from hydrogen production source 110 is passed directly to bioreactor 142. Microbial fermentation of CO in the presence of H₂ can lead to substantially complete carbon transfer into a product such as an alcohol, but, in the absence of sufficient H₂, only a portion of available CO is converted into product, while another portion is converted to CO₂ as in the following equation: 6CO+3H₂O→C₂H₅OH+4CO₂. Therefore, providing sufficient hydrogen to bioreactor 142 may be beneficial in some embodiments. In another embodiment, optional additional stream comprising CO₂ 440 generated from the gas production source 220 is passed directly to bioreactor 142. Such an arrangement may be beneficial to maintaining CO₂ partial pressure at CO₂ depleted zones of bioreactor 142.

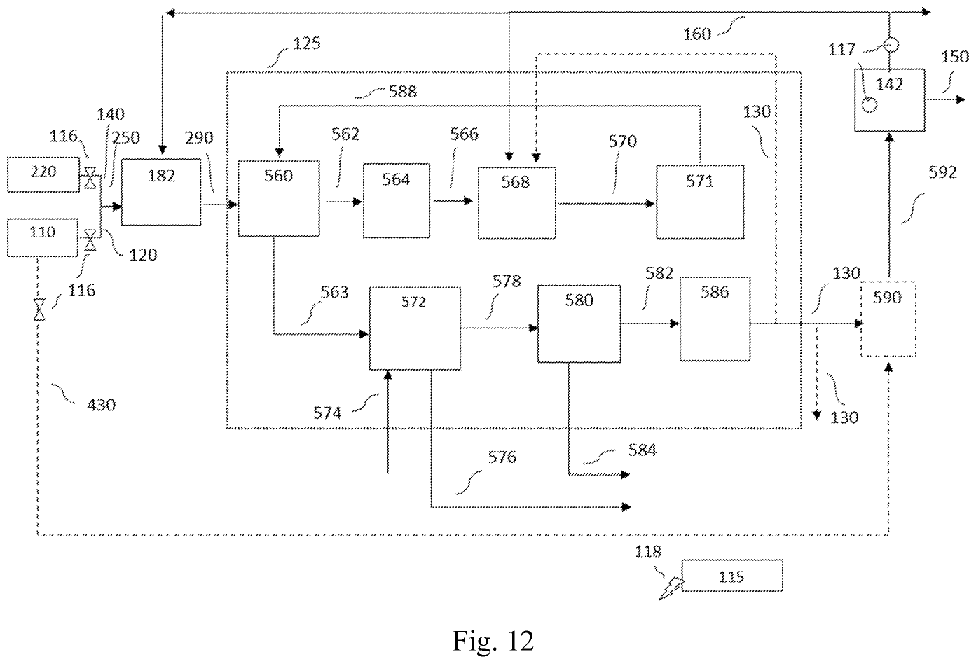

FIG. 12 is directed to an embodiment wherein the CO₂ to CO conversion system 125 is selected to be a rWGS system and additional equipment of the rWGS system is particularly depicted. Hydrogen production source 110 and first gaseous stream 120, as well as gas production source 220 and second gaseous stream comprising CO₂, and combined feed stream 250 are all described above. Gas treatment zone 182 and treated stream 290, plus bioreactor 142, fermentation product stream 150 and tail gas stream 160 are described above.

Treated stream 290 is introduced to preheater 560 where it is heated through indirect heat exchange with rWGS reactor effluent 588 to provide preheated stream 562. Preheated stream 562 is passed to electric heater 564 for further heating to generate electrically heated stream 566 which in turn is yet further heated in fired heater 568 to generate fully heated stream 570. Different modes of heating are employed to make the best use of available energy to arrive at a target temperature for the rWGS reactor. Heat in streams that need to be cooled is transferred to streams that need to be heated, and waste combustible components are burned in burners thus generating heat to heat streams needing elevated temperatures.

Fully heated stream 570 is introduced to rWGS reactor 571 which may be a single stage or multistage reactor system. In rWGS reactor 571, at least a portion of the CO₂ present in fully heated stream 570 is converted to CO. Thus, rWGS reactor effluent 588 is enriched in CO as compared to fully heated stream 570. Since rWGS reactor effluent is at the temperature of rWGS reactor 571, it contains available heat that may be used to heat another stream and is therefore passed to preheater 560 to indirectly heat exchange with treated stream 290. Heat exchanged rWGS reactor effluent 563 is then passed from preheater 560 to heat recovery/steam generator 572 to further recover available heat. Cool water stream 574 is passed to heat recovery/steam generator 572 to receive exchange of available heat from heat exchanged rWGS reactor effluent 563 and generate steam stream 576 which may be used elsewhere in the overall process or in another process. Resulting heat depleted stream 578 is passed to water knock out unit 580 to generate stream comprising water 584 and water depleted stream 582. Steam comprising water 584 may be directed to any portion of the process or another process needing water. Water depleted stream 582 is passed to air cooler 586 to provide CO enriched exit stream 130.

CO enriched exit stream 130 may be divided into portions, a first portion maybe passed to optional mixer 590, or when optional mixer 590 is not present, the first portion may be passed to bioreactor 142. An optional second portion of CO enriched exit stream 130 may be passed to another unit such as a buffer tank (not shown) or to an inoculator reactor that may or may not be part of bioreactor 142. Having stored amounts of CO enriched exit stream 130 is advantageous for time periods where the supply of gaseous stream comprising CO₂ is reduced. Where an inoculator reactor has lower hydrogen requirements as compared a bioreactor, passing a second portion of CO enriched exit stream 130 to the inoculator before addition of any additional hydrogen to of CO enriched exit stream 130 may be advantageous. An optional third portion of CO enriched exit stream 130 may be recycled to fired heater 568 to be combusted in the burners of fired heater 568 and provide heat. This embodiment is particularly advantageous at start up when bioreactor 142 is not yet on stream for consumption of the CO in the CO enriched exit stream 130.

In some embodiments it is advantageous to adjust and control the amount of hydrogen provided to bioreactor 142 by providing additional stream comprising hydrogen 430 from hydrogen production source 110 which is passed to mixer 590. In mixer 590, CO enriched exit stream 130 is mixed with additional stream comprising hydrogen 430 to generate bioreactor feed stream 592. The ratio of additional stream comprising hydrogen 430 from the hydrogen source to the CO enriched exit stream 130 is from about greater than 0:1 to about 4:1. Bioreactor feed stream is provided to bioreactor 142 and fermentation product stream 150 is produced as well as bioreactor tail gas stream 160. Bioreactor tails gas stream 160 may be divided into portions and recycled to different locations within the process. Where to route bioreactor tail gas often depends upon the current state of operation of the process. For example, when bioreactor 142 is operated in a mode that generates substantial CO₂, bioreactor tail gas 160 may have at least a portion recycled to gas treatment zone 182 or to CO to CO₂ conversion system 125 for conversion of CO₂ to CO. At any time, a portion of the bioreactor tail gas 160 may be supplied to the burners of fired heater 568 for combustion and generation of heat. Such use of at least a portion of bioreactor tail gas 160 for combustion is particularly advantageous in embodiments where bioreactor tail gas 160 contains methane. It is envisioned that biogas from a wastewater treatment system may be combined with bioreactor tail gas 160 and used for combustion and heat in fired heater 568. It is further envisioned that biogas from a wastewater treatment system may be recycled, or directly recycled to the bioreactor.

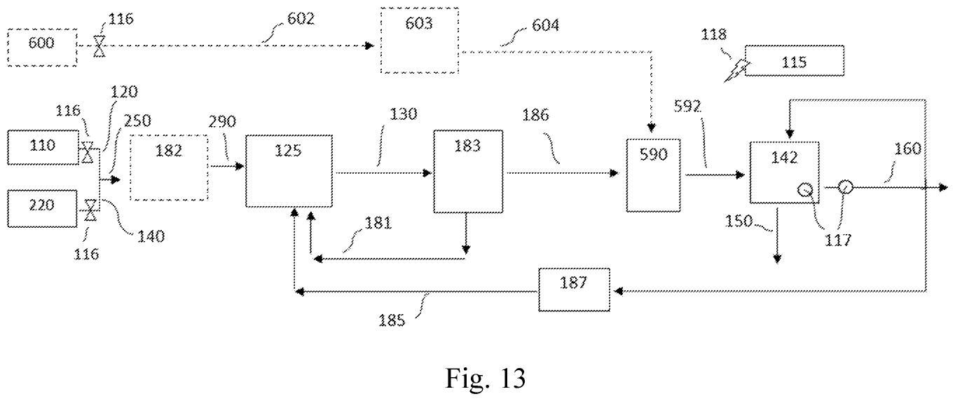

FIG. 13 is directed to an embodiment wherein a separate hydrogen stream is not passed through the CO to CO₂ conversion system but is mixed in downstream of the CO to CO₂ conversion system to form the feed stream to the bioreactor. Separate hydrogen stream 602 may be obtained from separate second hydrogen source 600 (as shown) or may be obtained from hydrogen source 110. Separate hydrogen stream 602 comprising hydrogen may be passed to optional hydrogen stream gas treatment zone 603 to produce treated hydrogen stream 604 comprising hydrogen. Hydrogen stream gas treatment zone 603 may include a gas component removal unit and or a gas desulfurization/acid gas removal unit. Both units may not be required in all embodiments, and hydrogen gas treatment zone 603 may contain only one of a gas component removal unit or a gas desulfurization/acid gas removal unit. Furthermore, the units in hydrogen stream gas treatment zone 603 may be in any order. Treated hydrogen gas stream 604 generated from hydrogen stream gas treatment zone 603 is passed to mixer 590 and mixed with treated CO enriched exit stream 186 to generate bioreactor feed stream 592.

Hydrogen production source 110, first gaseous stream comprising hydrogen 120, gas production source 220, second gaseous stream comprising CO₂ 140, and combined feed stream 250 are all described above. Gas treatment zone 182 and treated stream 290, plus CO to CO₂ conversion system 125, CO enriched exit stream 130, mixer 590, mixed stream 592, bioreactor 142, fermentation product stream 150 and tail gas stream 160 are described above but potentially with different ratios of H₂ and CO₂. Second gas treatment zone 183 and third gas treatment zone 187 are as described for gas treatment zone 182.

Turning to first gaseous stream comprising hydrogen 120 from hydrogen production source 110 and second gaseous stream comprising CO₂ 140 from gas production source 220, different ratios of hydrogen and CO₂ in the streams are useful at different points in the operation of the overall process. For example, the molar ratio of H₂ in first gaseous stream comprising hydrogen 120 to CO₂ in second gaseous stream comprising CO₂ 140, H₂:CO₂, may be about 1:1 in one embodiment, about 2:1 in another embodiment, and about 3:1 in still another embodiment. In the 1:1 H₂:CO₂ molar ratio embodiment, first gaseous stream comprising hydrogen 120 may have twice the volume of separate hydrogen stream 602 obtained from separate second hydrogen source 600. In the 2:1 H₂:CO₂ molar ratio embodiment, first gaseous stream comprising hydrogen 120 may have half the volume of separate hydrogen stream 602 obtained from separate second hydrogen source 600. In the 3:1 H₂:CO₂ molar ratio embodiment, first gaseous stream comprising hydrogen 120 provides all hydrogen needed and separate hydrogen stream 602 obtained from separate second hydrogen source 600 is not employed. Effectively, different amounts of hydrogen may bypass CO to CO₂ conversion system 125 through use of hydrogen stream 602/treated hydrogen gas stream 604. In one embodiment, the sum of hydrogen in first gaseous stream comprising hydrogen 120 plus hydrogen in separate hydrogen stream 602 provides sufficient hydrogen to yield a 3:1 molar ratio of H₂:CO₂ wherein the CO₂ is measured in second gaseous stream comprising CO₂ 140.

Tail gas stream 160 may be recycled to bioreactor 142 or recycled to CO to CO₂ conversion system 125. Optionally tail gas stream 160 may be passed through third gas treatment zone 187 to generate treated tail gas stream 185 which is then passed to CO to CO₂ conversion system 125. Second gas treatment zone 183 may optionally separate a portion of CO enriched exit stream 130 which can be recycled as stream 181 to CO to CO₂ conversion system 125.

FIGS. 1-13 further depict elements of the control system of the disclosure. One or more sensors 117 are used to measure the H₂:CO₂ molar ratio of the tail gas 160 of the bioreactor 142. Alternatively, or in addition, one or more sensors 117 are used to measure the H₂:CO₂ molar ratio of the headspace of the bioreactor 142. Sensors 117 may be analytical instruments such as gas chromatographs, probes, indicators, or other such measuring devices. Measurements providing the H₂:CO₂ molar ratio from sensors 117 are input into controller 115 using a wireless connection 118 or a wired connection (not shown). The controller may be a feedback loop controller. Controller 115 may be a distributed control system (DCS) type controller. Within controller 115, measured data providing the H₂:CO₂ molar ratio from sensors 117 are compared to a predetermined H₂:CO₂ molar ratio. Predetermined H₂:CO₂ molar ratio is selected by an operator and is based on a large number of variables. It is expected that predetermined H₂:CO₂ molar ratio would be different for different operations. Controller 115 would then operate to adjust the flow rates of first gaseous stream 140, second gaseous stream 120, additional stream comprising hydrogen 430, or optional additional stream comprising CO₂ 440 (as shown in FIG. 11), or any combination thereof in response to the difference between the measured H₂:CO₂ molar ratio and the predetermined H₂:CO₂ molar ratio in order to maximize the concentration of inert components in the tail gas stream. Adjustments to flow rates may be accomplished using flow controllers 116 which receive wireless 118 or wired (not shown) signals from controller 115. Inert components may be those that do not participate in the fermentation or participate in negligible quantities. In this way, the ratio of the gas substrates provided to the bioreactor of the gas fermentation process are controlled. In another embodiment, the sensors 117 may measure data to provide the H₂:CO:CO₂ molar ratio of tail gas 160 and or the bioreactor headspace, and the control process may proceed as described above using the measured H₂:CO:CO₂ molar ratio and a predetermined H₂:CO:CO₂ molar ratio. The goal of the control is to maximize the concentration of inert components such as nitrogen or, depending upon the microorganism, methane in the tail gas stream and or bioreactor headspace. For example, in one embodiment, the target maximum of the concentration of inert components in the bioreactor tail gas stream or the bioreactor headspace is from about 70 vol-% to about 80 vol-%.

Sensors 117 may obtain data continuously or periodically and the frequency may change depending upon different situations such as the performance of the bioreactor, the stage of operation of the bioreactor, situations involving hydrogen source or Cl source, operating conditions, environmental conditions, and others. Similarly, the predetermined molar ratios may change over time as well. Predetermined or target molar rations may be adjusted depending on situations such as the performance of the bioreactor, the stage of operation of the bioreactor, situations involving hydrogen source or Cl source, operating conditions, environmental conditions, and the like.

Likewise, the frequency of the controller operating to adjust the flow rates of one or more streams in response to the difference between the measured molar ratios and the predetermined or target molar ratio in order to maximize the concentration of inert components in the tail gas stream may vary as well. In situations where operations are fluctuating rapidly, adjustments may need to be frequent, whereas in steady state operation, adjustments may be less frequent.

Examples of possible analytical instruments include gas chromatographs with various modes of detection and gas analysers such as non-dispersive infrared (NDIR), electrochemical, dew point, and thermal conductivity.

All references, including publications, patent applications, and patents, cited herein are hereby incorporated by reference in their entirety as if each reference were indicated individually as such. References cited in this specification are not an acknowledgement that the reference forms part of the common general knowledge in the field of endeavour in any country.

Recitation of ranges of values herein are merely intended to serve as a shorthand method of referring individually to each separate value falling within the range, unless otherwise indicated herein, and each separate value is incorporated into the specification as if it were individually recited herein. For example, unless otherwise indicated, any concentration range, percentage range, ratio range, integer range, size range, or thickness range is to be understood to include the value of any integer within the recited range and, when appropriate, fractions thereof (such as one tenth and one hundredth of an integer). Unless otherwise indicated, ratios are molar ratios, and percentages are on a weight basis.

All methods described herein can be performed in any suitable order unless otherwise indicated herein or otherwise clearly contradicted by context. The use of any and all examples, or exemplary language like “such as” provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein. Variations of those embodiments may become apparent to those of ordinary skill in the art upon reading the foregoing description, and employment of such variations as appropriate, is intended to be within the scope as the disclosure may be practiced otherwise than as specifically described herein. Accordingly, this disclosure includes all modifications and equivalents of the subject matter recited in the claims as permitted by applicable law. Moreover, any combination of the above-described elements in all possible variations thereof is encompassed by the disclosure unless otherwise indicated herein or otherwise clearly contradicted by context.

1. A method for continuously controlling a ratio of input gases provided to a bioreactor of a continuous gas fermentation process comprising:
a. providing a gas fermentation process comprising
i. a first gaseous stream comprising H₂ from a H₂ source;
ii. a second gaseous stream comprising CO₂ from an industrial or syngas process;
iii. a CO₂ to CO conversion zone in fluid communication with the second gaseous stream and optionally the first gaseous stream, and having a CO enriched effluent comprising CO and CO₂;
iv. at least one bioreactor having at least one C-1 fixing bacterium for gas fermentation in a nutrient solution, the bioreactor having an product stream comprising at least one product, an outlet gas stream comprising H₂, CO₂, and inert components, a headspace comprising H₂, CO₂, and inert components, or both, the bioreactor in fluid communication with the CO enriched effluent, optionally the first gaseous stream, optionally the second gaseous stream, or any combination thereof;
b. measuring a H₂:CO:CO₂ molar ratio of the bioreactor outlet gas stream or the bioreactor headspace, to provide a measured H₂:CO:CO₂ molar ratio;
c. inputting the measured H₂:CO:CO₂ molar ratio to a controller and comparing the measured H₂:CO:CO₂ molar ratio to a predetermined H₂:CO:CO₂ molar ratio; and
d. adjusting the flowrate of the first gaseous stream, the flowrate of the second gaseous stream, or both, in response to the difference between the measured H₂:CO:CO₂ molar ratio and the predetermined H₂:CO:CO₂ molar ratio to maximize the concentration of inert components in the bioreactor outlet gas stream.

2. The method of claim 1 further comprising:
e) compressing, in a first compressor, at least a portion of the first gaseous stream, at least a portion of the second gaseous stream, or any combination thereof to generate a compressed first gaseous stream, a compressed second gaseous stream, and/or a compressed combination first gaseous stream and second gaseous stream;
f) treating:
i. at least a portion of the first gaseous stream or the compressed first gaseous stream, or both; and at least a portion of the second gaseous stream or the compressed second gaseous stream, or both; or
ii. the compressed combination first gaseous stream and second gaseous stream;
in a gas treatment zone comprising a gas component removal unit, a gas desulfurization/acid gas removal unit, or both before passing the second gaseous stream and optionally the first gaseous stream to the CO₂ to CO conversion zone; and
g) recycling the outlet gas stream to the first compressor, the gas treatment zone, the CO₂ to CO conversion system, the first gaseous stream, the second gaseous stream, or the combination of the first gaseous stream and the second gaseous stream.

3. The method of claim 2 further comprising, combining with the CO enriched effluent stream, at least a portion of:
i. the treated stream; or
ii. the first gaseous stream; or
iii. the second gaseous stream; or
iv. the combination of the first gaseous stream and the second gaseous stream; or
v. the compressed first gaseous stream; or
vi. the compressed second gaseous stream; or
vii. the compressed combination first gaseous stream and second gaseous stream; or
viii. any combination thereof.

4. The method of claim 1 wherein the first gaseous stream comprising H₂ is passed to the bioreactor without passing through the CO₂ to CO conversion zone, the method further comprising
e) compressing the bioreactor outlet gas stream to generate a compressed bioreactor outlet gas stream;
f) passing at least a first portion of the compressed bioreactor outlet gas stream, in any order, to:
i. a gas desulfurization and/or acid gas removal unit; or
ii. a gas component removal unit; or
iii. both the gas desulfurization and/or acid gas removal unit and the gas component removal unit;
to generate a compressed treated bioreactor outlet gas stream;
g) recycling the compressed treated bioreactor outlet gas stream:
i. to combine with the first gaseous stream, the second gaseous stream, or a combination thereof; or
ii. to the CO₂ to CO conversion system; or
iii. to combine with the CO enriched effluent stream; or
iv. any combination thereof; and
h) optionally recycling a second portion of the compressed bioreactor outlet gas stream to combine with the CO enriched effluent stream or to the bioreactor.

5. The method of claim 1 further comprising combining at least a second portion of the first gaseous stream, at least a second portion of the second gaseous stream, or a combination thereof, with the CO enriched effluent stream.

6. The method of claim 1 further comprising passing at least at least a second portion of the first gaseous stream, at least a second portion of the second gaseous stream, or a combination thereof, to the bioreactor.

7. The method of claim 1 further comprising compressing any portions of the first gaseous stream, the second gaseous stream, or combinations thereof.

8. The method of claim 1 further comprising controlling the relative amounts of the first portion of the compressed outlet gas stream and the second portion of the compressed outlet gas stream using a control valve.

9. The method of claim 1 further comprising passing at least a portion of the outlet gas stream to an outlet gas CO₂ to CO conversion system selected from a reverse water gas shift unit, a thermo-catalytic conversion unit, a partial combustion unit, a plasma conversion unit, a gasification unit, or a reforming unit, to generate a CO enriched effluent stream and recycling the second CO enriched effluent stream to the bioreactor.

10. The method of claim 1, wherein the CO enriched effluent stream comprises a H₂:CO:CO₂ molar ratio of about 5:1:1, about 4.5:1:1, about 4.33:1:1, about 3:1:1, about 2:1:1, about 1:1:1; or about 1:3:1.

11. The method of claim 1, wherein the CO₂ to CO conversion system comprises at least one of a reverse water gas shift unit, a thermo-catalytic conversion unit, a partial combustion unit, a reforming unit, or a plasma conversion unit.

12. The method of claim 1, wherein the product stream comprises at least one fermentation product selected from ethanol, acetate, butanol, butyrate, 2,3-butanediol, lactate, butene, butadiene, methyl ethyl ketone, ethylene, acetone, isopropanol, lipids, 3-hydroxypropionate, isoprene, fatty acids, 2-butanol, 1,2-propanediol, hexanol, octanol, or 1-propanol.

13. The method of claim 1, wherein the hydrogen source comprises at least one of a water electrolyser, a hydrocarbon reforming source, a hydrogen purification source, a solid biomass gasification source, a solid waste gasification source, a coal gasification source, a hydrocarbon gasification source, a methane pyrolysis source, a refinery tail gas production source, a plasma reforming reactor, partial oxidation reactor, or any combination thereof.

14. The method of claim 1, wherein the industrial or syngas process is selected from at least one of a sugar-based ethanol production source, a first generation corn-ethanol production source, a second generation corn-ethanol production source, a sugarcane ethanol production source, a cane sugar ethanol production source, a sugar beet ethanol production source, a molasses ethanol production source, a wheat ethanol production source, a grain based ethanol production source, a starch based ethanol production source, a cellulosic based ethanol production source, a cement production source, a methanol synthesis source, an olefin production source, a steel production source, a ferroalloy production source, a refinery tail gas production source, a post combustion gas production source, a biogas production source, a landfill production source, an ethylene oxide production source, a methanol production source, an ammonia production source, mined CO₂ production source, natural gas processing production source, a gasification source, an organic waste gasification source, direct air capture, or any combination thereof.

15. The method of claim 1, wherein at least one Cl fixing bacterium is selected from Clostridium autoethanogenum, Clostridium ljungdahlii, or Clostridium ragsdalei.

16. A system for controlling a ratio of substrate gases provided to a bioreactor of a continuous gas fermentation process comprising:
a. a first gaseous stream comprising substrate H₂ from a H₂ source;
b. a second gaseous stream comprising substrate CO₂ from an industrial or syngas process;
c. a CO₂ to CO conversion zone in fluid communication with the second gaseous stream and optionally the first gaseous stream, and having an effluent comprising CO and CO₂;
d. at least one bioreactor having at least one C-1 fixing bacterium for gas fermentation in a nutrient solution, the bioreactor having an tail gas stream comprising H₂, CO₂, and inert components, a headspace comprising H₂, CO₂, and inert components, or both, the bioreactor in fluid communication with the effluent comprising CO and CO₂, optionally the first gaseous stream, optionally the second gaseous stream, or any combination thereof;
e. sensors in the bioreactor tails gas stream or in the bioreactor headspace or both, capable of measuring the H₂:CO₂ molar ratio or the H₂:CO:CO₂ molar ratio of the bioreactor tail gas stream, or the bioreactor headspace, and providing a measured H₂:CO₂ molar ratio or a measured H₂:CO:CO₂ molar ratio;
f. a controller configured to accept inputs of the measured H₂:CO₂ molar ratio or the measured H₂:CO:CO₂ molar ratio and compare the measured H₂:CO₂ molar ratio to a predetermined H₂:CO₂ molar ratio or compare the measured H₂:CO:CO₂ molar ratio to a predetermined H₂:CO:CO₂ molar ratio; and provide outputs to adjust the flowrate of the first gaseous stream, the flowrate of the second gaseous stream, or both, in response to the difference between the measured H₂:CO₂ molar ratio and the predetermined H₂:CO₂ molar ratio or in response to the difference between the in response to the difference between the measured H₂:CO:CO₂ molar ratio and the predetermined H₂:CO:CO₂ molar ratio to maximize the concentration of inert components in the tail gas stream.

17. The system of claim 16, further comprising outputs to an operating parameter of the CO₂ to CO conversion zone to increase or decrease the relative amount of CO in the effluent comprising CO and CO₂.

18. The system of claim 16, wherein the CO₂ to CO conversion system comprises at least one of a reverse water gas shift process, a CO₂ electrolyzer, a thermo-catalytic conversion process, a partial combustion process, or a plasma conversion process.

19. The system of claim 16, wherein the gas fermentation process further comprises a gas treatment zone in fluid communication with the first gaseous stream, the second gaseous stream, the effluent, or any combination thereof.

20. The system of claim 16, wherein the gas fermentation process further comprises at least one compressor in fluid communication with the first gaseous stream, the second gaseous stream, the effluent, or any combination thereof.

21. The system of claim 16, wherein the gas fermentation process further comprises a methane conversion zone in fluid communication with the bioreactor tail gas stream, the methane reforming zone comprising an effluent conduit in fluid communication with the CO₂ to CO conversion zone.
